(19) United States
(12) Patent Application Publication
(10) Pub. No.: US 2013/0061496 A1
(43) Pub. Date: Mar. 14, 2013

(54) FOOTWEAR SUPPORT STRUCTURES

(71) Applicant: Kevin B. Lawlor, Wellesley, MA (US)

(72) Inventor: Kevin B. Lawlor, Wellesley, MA (US)

(21) Appl. No.: 13/355,622

(22) Filed: Jan. 23, 2012

Related U.S. Application Data
(60) Provisional application No. 61/532,382, filed on Sep. 8, 2011.
(60) Provisional application No. 61/549,373, filed on Oct. 20, 2011.

(51) Int. Cl. A61F 5/14; A43B 3/12; A43B 3/00; A43B 5/00

(52) U.S. Cl. 36/114; 36/140; 36/11.5; 36/83

(57) ABSTRACT

Footwear as inserts to shoes or integral parts to shoes is made of rigid form with a sole portion to be placed in compression and an upper portion to flex in tension in response to foot motion and preferably is provided in a triangulation form for foot stabilization, comfort and injury prevention.

### FIELD OF THE INVENTION

The present invention relates generally to the field of footwear and footwear components. This application claims priority from U.S. Application Ser. No. 61/532,382 filed Sep. 8, 2011 and from U.S. Application Ser. No. 61/549,373 filed Oct. 20, 2011.

### BACKGROUND OF THE INVENTION

The modem athletic shoe was made possible by technological advances, including manufacturing innovations and new materials applied to footwear. These innovations in footwear combined with the notion of better health through exercise. The jogging trend of the 1980's introduced the modem athletic shoe to the consumer. A variety of color choices made athletic shoes popular fashion choices. Wearing modem athletic shoes, millions of people of all ages are able to enjoy running and other athletics for exercise, better health and pain relief.

The two main components of a manufactured shoe are the upper and the shoe sole. The shoe sole and the upper are typically made separately and joined by glue, and/or stitching and/or the material of the sole. Athletic shoes and running shoes in particular, have suede and/or a lightweight leather and cloth upper. The extremes of bodily motion occurring in sports require an upper portion that is malleable. However, a malleable upper changes its shape and becomes less able to contain the foot in its original position.

The most popular athletic shoes are running and basketball shoes. They are sold as specific to these sports, but are in common usage for all footwear purposes. Lateral moves in the game of basketball involve the ankles. Basketball shoes generally provide support above the foot due to their portions that lace above the ankle. Running and basketball shoes are the choice for young and old desiring a more cushioned footstep and ankle support respectively.

The foam portion of the running shoe sole provides cushioning, but takes a “compression set.” With repeated pressure the foam material collapses in a predictable pattern and its ability to cushion greatly diminishes. Over time, the combination of a malleable upper and a deforming cushioned sole decreases the structural integrity of this type of footwear. During walking and running the foot experiences increased pressure from the weight of the body. As a result, foot discomfort may occur. This; may be limited to general foot fatigue but can also lead to other adverse effects. The solution may be athletic shoes with cushioned soles. However, generalized foot fatigue may become localized resulting in pain in certain areas of the foot. When localized pain occurs, often the first response is to address the condition at its source. The pain may be diminished or overcome by placing foam or other cushioning material under the area of pain and/or under the whole foot. Many products are designed for different areas of foot pain and are available over the counter to consumers. Persistent foot pain may require medical help. A doctor's suggestion may be other than placing a soft cushion under the painful area, although a cushioning component may be added. Structures and supports of plastic material in the form of foot orthotics may be prescribed by a Podiatrist or doctor. They are most often added under the foot to be placed inside a shoe to limit discomfort.

Configured plastic materials are used in foot orthotics and also shoe counters. Similar plastic materials have been used in other areas of shoes including the soles of running and sports shoes. When structured appropriately to the foot and footwear these materials may retain some of the structural integrity missing in athletic shoes with malleable upper portions and foam material soles. Whether configured with structures to provide components for athletes, those experiencing foot discomfort and those without foot problems, the flexibility, strength and longevity of plastic material may lend itself to additional improvements to footwear and footwear manufacture.

### SUMMARY OF THE INVENTION

The present invention provides a footwear article construction, in a shoe, boot, sneaker, sandal or the like product or in a form integral to such product or as an insert for any such product, that differs from others of such products or inserts in that the following structural features to apply the structural elements of cooperative compression and tension to manage triangulation of the foot in motion. These structural features utilize the downward pressure of the foot in motion to deliver its sideways directional force to the structure which is returned to the foot by reaction forces, thereby providing comfortable fit (of the shoe etc.) as a while, improved athletic or rigorous walking performance and eliminating or minimization of disabling expensive and/or generalized or local foot discomfort. The sideways directional force creates tension in the footwear that provides shock absorption to the foot in motion. The material of the invention is structurally engineered with features designed for the absorption of the directed force to specific areas of the foot. It achieves this process with the stepping action of a footstep.

Compression holds the footwear in position. The footwear in turn, sustains the foot. In several of the embodiments described herein, compression is followed by tension which may include limited flexing of an upper vertical portion. Tension results in shock absorption to the foot in motion. Portions of the invention are held in position by compressive downward pressure that is greater than sideways directional force. Momentum of the sideways directional force is absorbed by other portions of the footwear. Momentum in those other portions creates tension in the footwear to provide shock absorption to the foot in motion.

In the present invention, a side substantially vertical medial portion is provided next to the first metatarsal with a bottom substantially horizontal medial portion attached underneath. The first metatarsal in the forefoot area just ahead of the arch (mid-foot) portion is an area of triangulation in the foot. The downward pressure of compression is greater than sideways directional force so the horizontal medial bottom portion remains relatively in position while the vertical side support can flex in tension. With the horizontal bottom portion in compression and in position, the medial vertical side support is in tension and can flex, absorbing the directional force of momentum to provide shock absorption to the foot in motion.

Further, similar shock absorption can occur on the lateral side. The fifth metatarsal is another area of triangulation in the foot. Further, some embodiments of the invention place the more mobile first metatarsal portion in tension in relation to the position of the less mobile fifth metatarsal located across from the first. The further benefit of this footwear construction is cross foot tension in addition to medial and lateral side tension.

Further, triangulation occurs naturally in the foot to stabilize it. Rear foot impact is the initial point of a foot's triangulation. Whether or not the footwear is present, outward triangulation forces are initiated and if footwear is constructed as shown in the present invention response forces are created to manage the triangulation. In the invention, triangulation from the foot is managed to the invention's rear (hind foot) portion of the footwear, with or without rear foot verticals, to the mid foot/forefoot vertical lateral side portion, and to the medial forefoot side portion and stabilizes the footwear and holds it in position. The triangulation of the footwear interacts with the continuing action of the foot's triangulation to contain movement of the foot and thereby sustaining it.

Rear foot, mid foot and forefoot footwear triangulations manage the foot in motion. Mid-foot and forefoot triangulations of footwear perform similar functions. Forefoot portions of footwear triangulate performing similar functions. Forefoot only footwear with and without triangulations are in compression and tension. Further, forefoot footwear do not require a portion over the top of the foot and can perform independent of them.

Compression and tension are further utilized in an effective manner for redirection and shock absorption when a shape similar to a convex leaf spring is applied to footwear in the rear and forefoot and medial side arch. Through additional responses to compression these embodiments may increase shock absorption. These structures may be located in the shoe sole and upper of footwear. Their location and the location of other structural features of the invention may unite into a structure of similar materials around which manufacture of footwear can occur.

Like other relatively sturdier materials that underlie and bear weight and forces rather than appear this structure too can support the whole foot in motion whether appearing in portions on the surface of footwear or underlying. Embodiments of the upper, sole and of the footwear as a whole may include attachments. These and other novel features of the invention, including the ability to integrate similar material and structures in the upper with the shoe sole are contributions to the state of the art of footwear.

There is a difference between a directed shock absorbing structural element and an all over area of cushioning foam. Unlike foam that can only be layered in different densities, plastic and plastic like material can be designed and molded to include structural features in footwear and manage triangulation.

Other objects, features and advantages will be apparent from the following detailed description of preferred embodiments taken in conjunction with the accompanying drawings in which:

### BRIEF DESCRIPTION OF THE DRAWINGS

FIG. 1 displays a footprint and force arrows related to the present invention;

FIG. 2 displays a top view of a footprint, bones of a foot and force arrows;

FIG. 3 displays a medial side view of an embodiment of the present invention;

FIG. 3A displays a top view of an embodiment of the present invention;

FIG. 3B shows a side view of the bone structure of a foot and an embodiment of the invention;

FIG. 3C shows a top view of the bone structure of a foot and an embodiment of the invention;

FIG. 4 shows a side view of a larger embodiment of the invention relative to a shoe counter and insole;

FIG. 4A shows a top view of a larger embodiment of the invention relative to a shoe counter and insole;

FIG. 5 displays a side view of an embodiment of the invention comprising a counter portion;

FIG. 5A displays a top view of an embodiment of the invention comprising a counter portion;

FIG. 6 displays a top view of an alternate embodiment of the invention;

FIG. 7 displays a top view of an alternate embodiment of the invention;

FIG. 8 displays a top view of an alternate embodiment of the invention comprising a bridge and cushioning;

FIG. 9 displays a top view of an alternate embodiment of the invention comprising a bridge and cushioning;

FIG. 10 illustrates a top view of an alternate structure of the invention;

FIG. 11 illustrates a top view of an alternate structure of the invention including a means for adjustment;

FIG. 12 displays a top view of an alternate structure of the invention including a more rearward positioned bridge and rear wings;

FIG. 13 illustrates a top view of another alternate structure of the invention including a means for adjustment;

FIG. 14 displays a top view of an alternate embodiment of the invention.

FIG. 15 illustrates a top view of an embodiment of the invention including a means for adjustment;

FIG. 16 illustrates a top view of an embodiment of the invention including a means for adjustment;

FIG. 17 illustrates a top view of an embodiment of the invention including a means for adjustment;

FIG. 18 illustrates a top view of an embodiment of the invention including a means for adjustment;

FIG. 19 illustrates a top view of an embodiment of the invention including a means for adjustment;

FIG. 20 illustrates a top view of an embodiment of the invention including additional means for adjustment;

FIG. 21 illustrates a top view of an embodiment of the invention including additional means for adjustment;

FIG. 22 illustrates a top view of an embodiment of the invention including a means for adjustment and placement of the invention;

FIG. 23 illustrates a top view of an embodiment of the invention including a means for adjustment and placement of the invention;

FIG. 24 illustrates a top view of an embodiment of the invention including a single portion;

FIG. 24A illustrates a top view of an embodiment of the invention including a top of the foot portion;

FIG. 25 illustrates a top view of an embodiment of the invention including a single portion;

FIG. 25A illustrates a top view of an embodiment of the invention including a top of the foot portion;

FIG. 26 illustrates a top view of an embodiment of the invention including additional means for adjustment;

FIG. 26A illustrates a top view of an embodiment of the invention including a top of the foot portion;

FIG. 27 illustrates a top view of an embodiment of the invention including additional means for adjustment;

FIG. 27A illustrates a top view of an embodiment of the invention including a top of the foot portion;

FIG. 28 illustrates a top view of an embodiment of the present invention;

FIG. 28A illustrates a cut-away view of a portion of FIG. 28;

FIG. 29 illustrates a top view of an embodiment of present invention;

FIG. 29A illustrates a cut-away view of a portion of FIG. 29;

FIG. 30 illustrates a top view of an embodiment of the invention including polygon shaped adjustment portions;

FIG. 30A illustrates a cut-away view of a portion of FIG. 30;

FIG. 31 displays a top view of a footprint, bones of a foot and placement of cutaways in relation to FIGS. 32, 33, 33A;

FIG. 32 illustrates a cutaway of a convex form with a strike plate in the rear foot area of footwear shown in FIG. 31;

FIG. 33 illustrates a cutaway of a convex form with a strike plate in the forefoot area of footwear shown in FIG. 31; and

FIG. 33A illustrates an enlargement of cutaway FIG. 33 of a convex form with a strike plate in the forefoot area of footwear shown in FIG. 31.

### DETAILED DESCRIPTION OF THE INVENTION

The following terms, as used relative to the present invention have the meanings as listed below:

Glossary
Sustain—To keep something in position by holding it from below
Compression—Pushing, downward pressure
Tension—pulling, pulling apart
Vector—directional force, possessing both magnitude and direction represented by an arrow
Force—something that changes the motion of an object (speed-momentum and/or direction)
Impact—rear of the foot striking the ground during a footstep
Medial—extending towards the middle of the body
Lateral—extending toward the outside of the body
Momentum—the product of mass and velocity
Tensile strength—maximum stress a material can handle when being pulled or pushed
Elastic deformation—self reversing shape change.
Strike plate—a material platform sustaining the downward pressure of the sides of a convex shape.

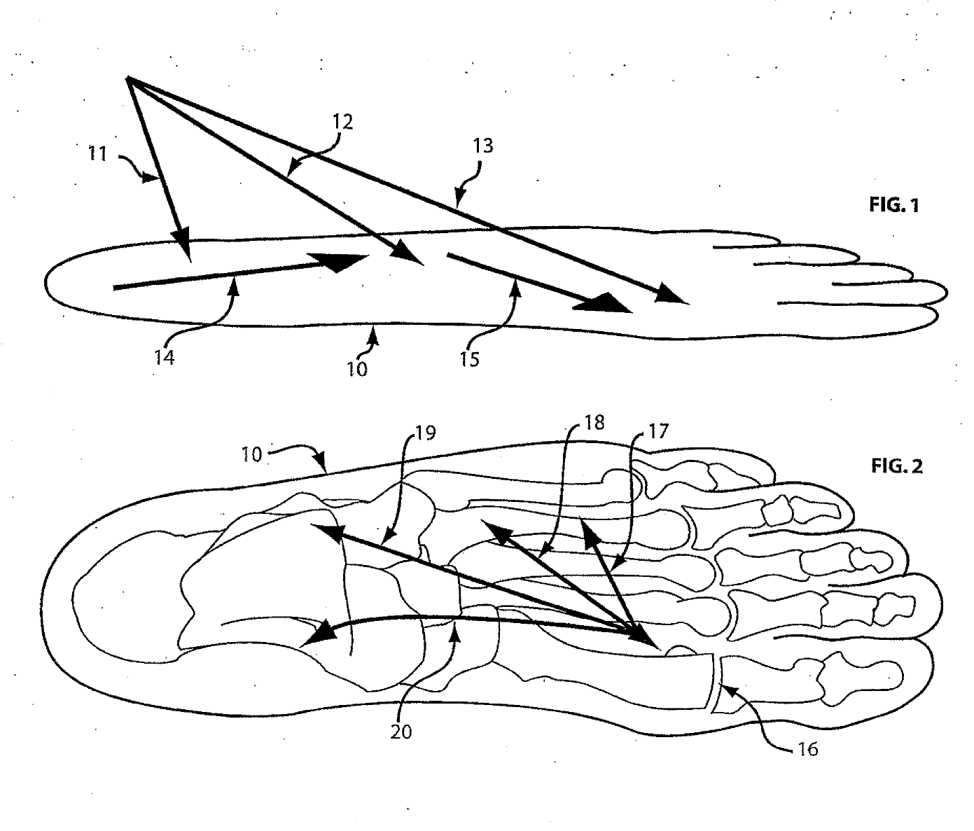

There is shown, in FIGS. 1-33A improvements to footwear. Embodiments of the invention, shoe components and footprint and foot are illustrated for a user's left foot, but it should be understood that an analogous structure and analogous functions may be exhibited by a right foot and shoe components made according to the invention.

The present invention utilizes downward pressures indicated in FIG. 1 by vectors 11, 12, 13 on footwear to sustain the foot in motion and sideways directional forces indicated in FIG. 1 by vectors 14, 15 to absorb shock, brace and support the foot. The invention uses these vectors of a foot in motion to mollify lateral and medial pressures and forces on the foot. Downward pressures 11, 12, 13 are greater in magnitude than sideways directional forces 14, 15. This is used to advantage to create tension in the invention. Preferred practice of the invention is to engage the lesser directional force and momentum placed on the foot by vectors 14 and 15 to absorb shock, brace and support the foot in motion and thereby manage triangulation.

The quality of compression is pushing and tension is pulling. Compression activates the invention. The action of applying downward pressure in relation to directional force initiates tension within the invention. Tension in the invention provides support for the foot in motion.

The implementing structure per the present invention may be located substantially inside shoes in preferred embodiments or integral with shoe construction in others. The invention can be attached to shoes during manufacture or can be inserted into a shoe by the consumer. The invention can be made by a manufacturer, a podiatrist, a doctor for a patient and those familiar with constructing footwear orthotics.

FIG. 1, 10 show a footprint. Downward facing arrows of vector 11 at the rear of the foot, 12 at the mid and forefoot and 13 at the forefoot illustrate, in sequence the downward pressure placed on a foot during a footstep. The rear area of the foot bears the greatest impact during forward motion. During activities like running, impact there can be three times body weight. During walking it is less. On the footprint 10 in FIG. 1 two vectors 14, 15 indicate the sequence of downward pressure and directional force moving forward. As the impact to the rear of the foot abates, pressure and force move forward and to the lateral side vector 14 at mid foot and forefoot. Vector 15 indicates the direction of force and pressure in the footprint 10 in FIG. 1 from the lateral to the medial side of the foot toward the big toe and joint of the forefoot FIG. 2, 16. The medial side being the side towards the center of the body and the lateral is the side towards the outside of the body, opposite from the medial.

Forward heads of the two-headed directional arrows of FIG. 2, noted as vectors 17, 18, 19, 20 on the footprint 10 indicate the lateral and medial areas of directional force and downward pressure directed to the forefoot and the big toe and joint of the forefoot 16. While rear facing arrows of vectors 17, 18, 19, 20 indicate the origins of the pressure. Rear facing heads of two-headed directional arrows vectors 17, 18, 19 on the lateral side and vectors 20 on the medial side also indicate the oppositional force from the foot and the invention back from the forefoot to the lateral side forefoot, mid foot and rear foot.

Downward pressures and directional forces of a foot in motion in FIG. 2, vectors 17, 18, 19 proceed forward and all three converge on the forefoot medial side on or near the joint 16. On the medial side, weight moves from the rear portion of vector 20 to the forefoot portion of vector 20. Referring to FIG. 2, vector 20 there is height, or bridging, in the area of the arch, and relative time off the surface on the medial side in relation to the incremental less bridged movement of the lateral. Medial height/bridging and relative time off the surface, allows for pivoting of direction to the medial side, but also more downward pressure at the forefoot as the transfer of weight to the opposite foot also begins. Forefoot medial area is an important area as regards the invention because of the confluence of downward pressures, directional forces, and the completion of triangulation just before the big toe pushes off and weight is transferred to the opposite foot. Therefore, all areas whether or not there is concomitant footwear, participate in the triangulation. Footwear of the invention may include components in this area.

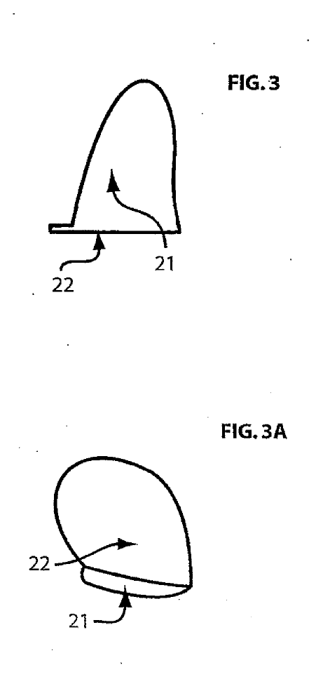

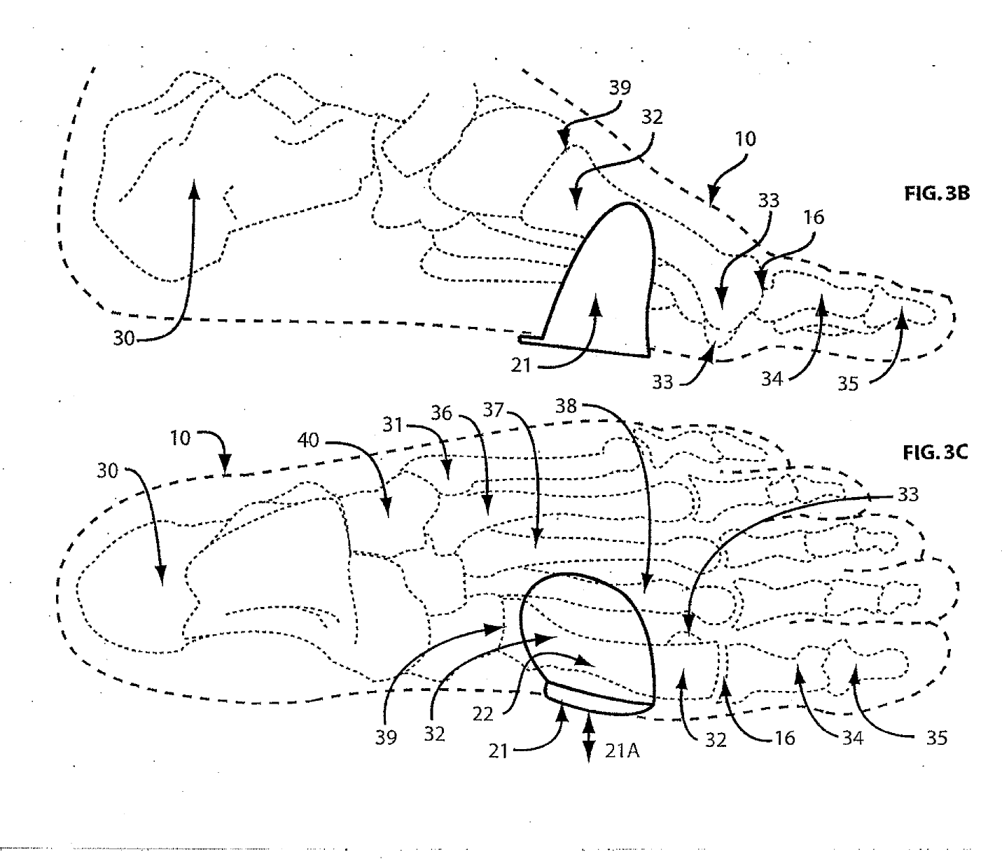

FIG. 3 shows a medial side view and FIG. 3A a top down view of an embodiment. This embodiment of FIGS. 3 and 3A comprises an angled element. Vertical medial side support 21 is a medial substantially vertical side support that may flex when in tension with a medial substantially horizontal medial bottom portion 22 that remains relatively in position during the downward pressure of the foot in motion. With downward pressure portion 22 is in compression and 21 is in tension. Portion 21 is the upper of the element and 22 the sole. The area 21 may experience elastic deformation in footwear. Maximum stress may occur there in footwear due to the confluence of forces, pressures and weight transfer. As a result, the material design for the particular application may require a tensile strength test and an analysis of elastic deformation obtained to see how much the first metatarsal is restrained but to see at least some flexing movement. Compression occurs elsewhere in the sole and tension elsewhere in the upper of footwear. All areas of compression and tension in footwear may require similar testing. Vertical medial side support 21 can be located inside as well as outside of footwear. Horizontal medial bottom portion 22 can be inside as well as outside of footwear as well. It can be attached to bottom sole plastic material currently visible in some footwear. It may be located anywhere between the bottom outside of footwear and the inside of footwear.

In the embodiment of FIGS. 3B, 3C bones of the foot are shown. Beginning in the rear foot at impact the calcaneus 30 is shown, 40 is the cuboid bone, mid foot on the lateral side, the fifth metatarsal 31 is forward of the cuboid, moving toward the medial side are metatarsals four 36, three 37, two 38, one 32, sesamoid bones 33, and phalanges 34, 35 of the big toe. Joints of the first metatarsal 32, 39, 16 are also shown. The calcaneus 30 in the rear foot bears the initial impact of foot-strike. Forward of the calcaneus 30, pressure on the foot varies as the force moves forward and towards the lateral side and the area of the fifth metatarsal 31, vector 14 in FIG. 1 and then to the medial side of the foot and the area of the first metatarsal 32, vector 15 in FIG. 1. The three bones, calcaneus 30, fifth metatarsal 31, and first metatarsal 32 form a triangle, a stable form. In preferred embodiments, the invention responds and interacts with forces in these three areas: the calcaneus 30, the fifth metatarsal 31 and the first metatarsal 32, sequentially as the directional forces change. Adjoining areas also triangulate.

FIG. 3B shows a side view and FIG. 3C shows a top view of the bone structure and an outline of the foot 10 both in dashed lines. It shows the placement of vertical medial side support 21 and horizontal medial bottom portion 22 of the invention in relation to the bones of the foot. Vertical medial side support 21 is beside, the first metatarsal. In alternate embodiments vertical medial side support 21 may be further rearward at or towards the joint 39 or forward towards joint 16. Further, vertical medial side support 21 may be an upside-down and backwards “L” shape with the top backwards rear foot facing portion extending over to the joint 39 and maybe slightly beyond. The horizontal medial bottom portion 22 is under the first metatarsal; the largest and thickest bone in the forefoot. The downward pressure and lateral to medial directional force on the foot in motion puts portion 21 in tension. The downward pressure on the horizontal medial portion 22 is greater than the lateral to medial sideways directional force on portion 21. As a result, under the foot portion 22 stays in position sustaining the foot. Portion 21 in relation to portion 22 may or may not flex in tension as directional force vector arrow 21A, pointing from the lateral to the medial side, is applied to the first metatarsal 32 of the foot. The response of portion 21 is indicated by the other directional force vector arrow 21A, pointing from the medial to the lateral side, on the first metatarsal 32. Whether portion 21 flexes or not, the response of the tension in portion 21 is to absorb momentum of the directional force of the foot in motion. As a result, the invention absorbs shocks, braces, supports, and stabilizes the first metatarsal and other bones and muscles of the foot in motion.

The footstep continues past this forefoot area. The ball of the foot rolls further towards the medial side and the forefoot big toe. Downward pressure moves to the vertical medial side support 21 from the horizontal medial bottom portion 22. Portion 21 briefly pulls portion 22 vertically providing relief from downward pressure there. It provides support and lessens the downward pressure on portion 22 just after it is at its greatest.

In footwear the horizontal area of horizontal medial bottom portion 22 may be extended towards the lateral area of the foot along vectors 17, 18 in FIG. 2 which in turn may increase the area of downward pressure and increase the likelihood of portion 22 remaining in position while directional force is directed toward vertical portion 21. Sole portion 22 of footwear may extend further towards the rear foot encompassing areas of vectors 19, 20 as well.

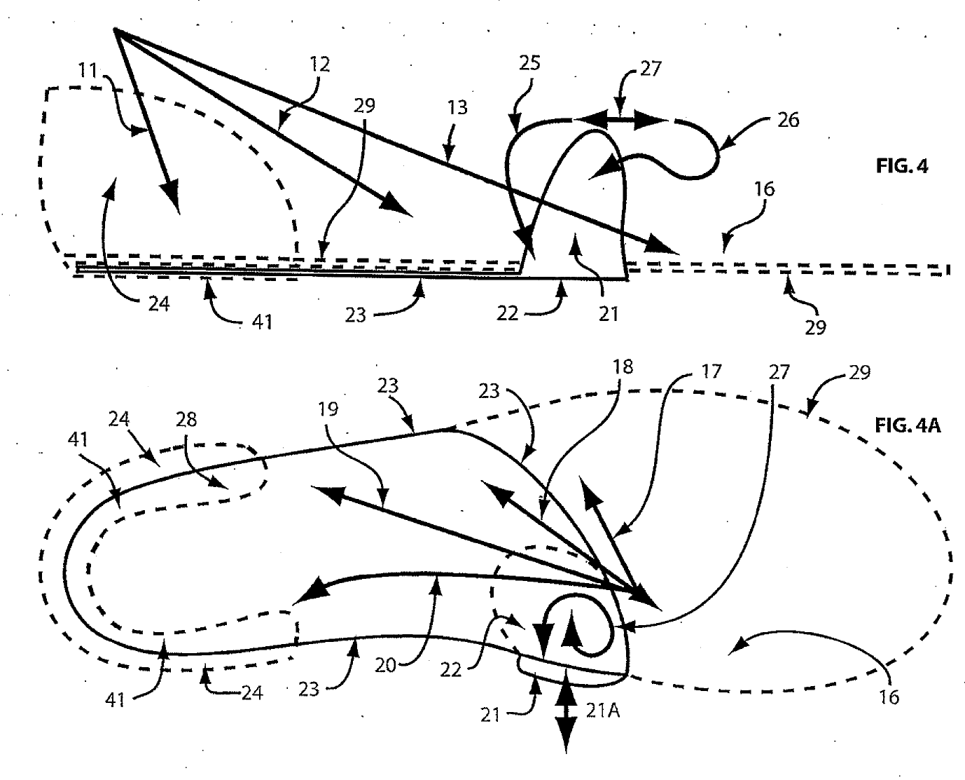

In the embodiment of FIGS. 4, 4A the invention provides a structure in the upper forefoot vertical medial side support 21, and horizontal medial bottom portion 22 in the sole that may be made of a similar material to the vertical shoe counter 24, and lateral and medial side horizontal bottom shoe counter 28, 41. Although the material of the counter 24, 41 is unattached to embodiment FIGS. 4, 4A its placement may engender tension and compression. Similar proximity in footwear may also bring about tension and compression and triangulation of footwear of the invention. Therefore the upper portion may include not only 21, but also 24, 41 in tension and not only 22, 23, but also 41, 28 in compression.

In the embodiment of FIGS. 4, 4A horizontal support 22 is increased in size. It extends towards the lateral area of the foot along vectors 17, 18 and to the rear foot areas vectors 19, 20. The increase of the size of the substantially horizontal portion of the embodiment from 22 to 22, 23 in turn increases the downward pressure and makes the embodiment more likely to remain in position. The stability resulting from the increase in size and downward pressure allows portion 21 to be braced incrementally more consistently. It is thought a pain free consistent repetitive foot-strike is advantageous. A consistent repetitive foot-strike provided by embodiment FIGS. 4, 4A may provide consistent support and bracing for the first metatarsal.

Side view FIG. 4 shows the vertical medial side support 21 and horizontal medial bottom portion 22 connected to 23 making this embodiment 21, 22, 23. FIG. 4 shows this embodiment in relation to an insole 29 and extending back as far as the insole. FIG. 4A shows the embodiment following in part the outline of the insole 29. It is understood the footwear can align with, be above, below or part of an insole 29. FIG. 4, 4A with portions 21, 22, 23 can be an orthotic device added by the consumer to footwear or by the manufacturer.

Following impact, vertical medial side support 21 and horizontal medial bottom portion 22 experience downward pressure 11 from the rear foot area and downward pressures 12, 13 in the mid foot and forefoot area. Top view FIG. 4A shows vertical medial side support 21, and horizontal medial bottom portion 22 in the area of four two headed forward facing arrowhead vectors, one on the medial side 20 and three on the lateral side 17, 18, 19. Vertical medial side support 21, horizontal medial bottom portion 22 experience these directional force vectors 17, 18, 19, 20 to the forefoot medial side. FIGS. 4, 25, 26, 27 and FIGS. 4A, 27, 21A show this footwear in tension between its vertical medial side support 21 and horizontal medial bottom portion 22. The resulting tensions and possible flexing of vertical medial side support 21, and horizontal medial bottom portion 22, are accommodated by the flexibility of the material of the invention.

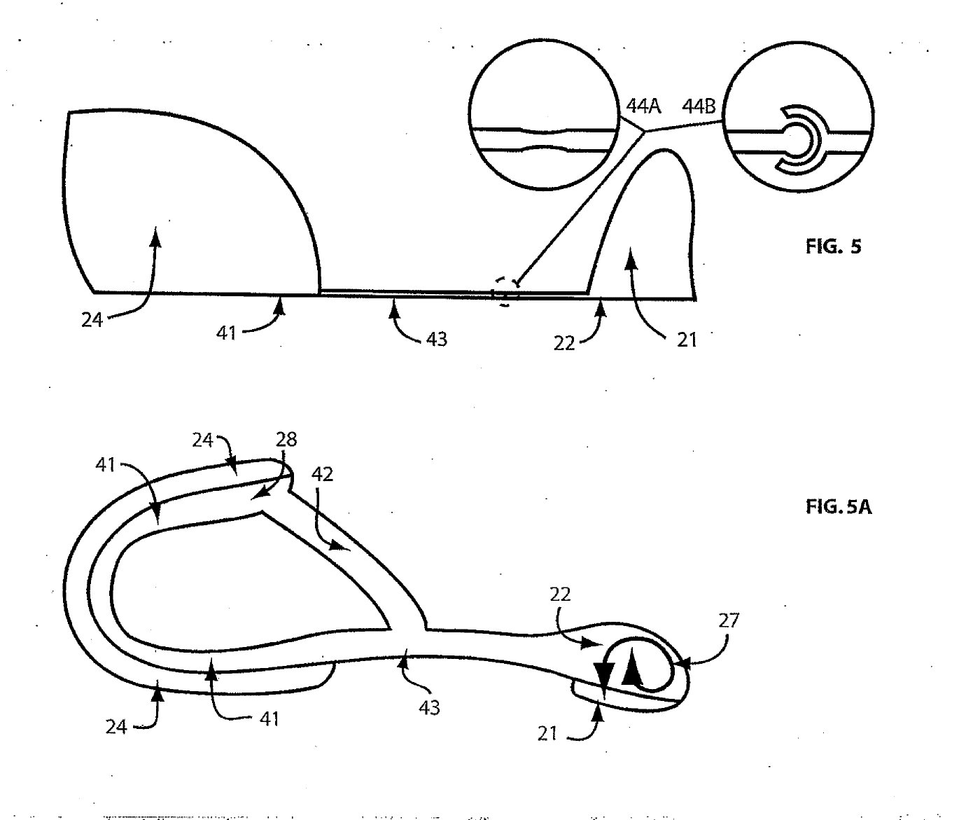

Tension and compression in vertical medial side support 21, and horizontal medial bottom portion 22 on the medial side in FIGS. 3, 3A, 3B, 3C, 4, 4A is also present in the embodiment of FIGS. 5, 5A, indicated by 27 in FIG. 5A, and also on the medial side of all other embodiments of the footwear. Material may be lessened in the sole of footwear to lighten it while it still remains sturdy. FIG. 5A shows a vertical counter 24 with lateral side horizontal bottom shoe counter portion 28, 41. The counter is joined around the rear of the foot 24, lateral side 41 to medial side 41 and forward to a mid foot horizontal connector area 43 and then to medial forefoot area and d 21, 22. The increase of area provided by horizontal counter portions 28, 41, 42, 43, 22 provides an increase of downward pressure on the footwear thereby increasing the likelihood of portion 22 remaining in position while directional force is directed toward vertical portion 21 and the first metatarsal 32. Vertical shoe counter portion 24 extending up around the heel from the lateral to the medial side stabilizes the rear foot and rear foot portion of footwear thereby assisting in holding the rest of the embodiment in position in relation to the foot. Therefore, footwear with 24, 41 and/or lateral 28 includes triangulation for the foot in motion.

Crossing from the lateral side of area 28 over to the medial side at or near 43 is a bridge 42. Connecting the lateral to the medial reinforces the structure and contributes to holding the embodiment in position. Both downward pressures in the middle of the foot and directional forces 19 act on connector 42 further stabilizing footwear. In other footwear a bridge may proceed from area 28 of the counter under the foot and over to area 22. This footwear may or may not include area 43.

Downward pressure affects this and other embodiments along the path of the two headed arrow 19 of FIG. 2 first from the lateral then to the medial side and then there are counteractive directional forces from the medial 19 to the lateral side. As a result, the upper of footwear in tension includes portions 21, 24 while the sole portion of footwear in compression includes 22, 28, 41, 42, 43.

FIG. 5 shows a call out 44A. Bending of the forefoot during forward motion may require the footwear to bend as well. Thinner in the middle, area 44A in FIG. 5, bends when the foot bends. Call out 44B shows a ball and socket joint allowing the footwear to bend at this point in a similar way. Placement of 44A and 44B are specific to the user and can be arrived at through testing. Other places in the footwear may require a bending mechanism. These may be used elsewhere in the invention. Other methods to bend the invention may also be used. Further, in alternate embodiments the footwear may not need an intentional bend and may adjust on its own due to the flexibility of the material.

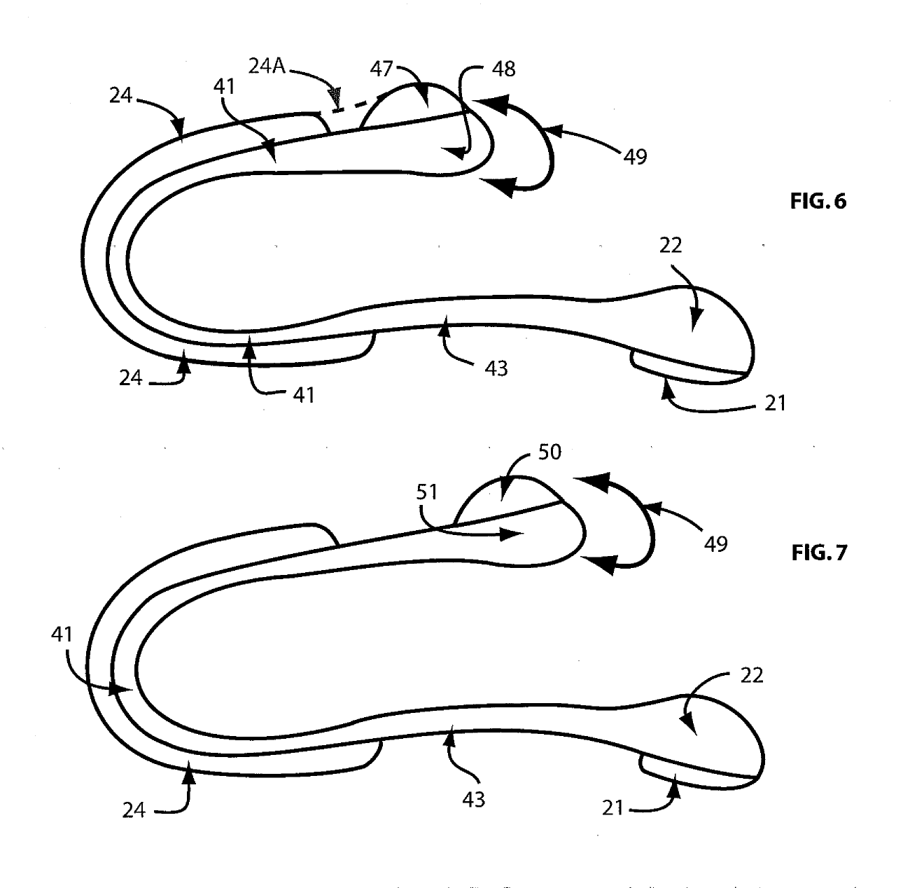

FIGS. 6, 7 show top views of two further embodiments of the invention attached in part to the counter 24, 41 of a shoe. In FIG. 6 forward lateral horizontal portion 48 of the embodiment continues farther forward than the lateral side counter 28, 41 in FIGS. 5, 5A. It shifts direction and continues up vertically 47 to rest beside the cuboid bone 40 in FIG. 3C. FIG. 7 portion 51 continues farther forward than the counter 28, 41 in FIGS. 5, 5A to underneath the rear base portion of the fifth metatarsal bone 31 in FIG. 3C and then up vertically 50 beside the fifth metatarsal bone 31. It is understood that these and other footwear can be independent of the shoe and counter. It is also understood the areas of 47, 48, 50, 51 may overlap in alternate embodiments. It is also understood in footwear dashed line 24A in FIG. 6 may connect the counter 24 to 47 and in FIG. 7 counter 24 may be connected to 50. The upper of footwear in tension includes portions 21, 24, 47, 50 while the sole portion of footwear in compression includes 22, 48, 51, 41, 43.

Vectors 17, 18, shown in FIG. 2, are closer to the fifth metatarsal, a point of triangulation of the foot. Triangulation provides an angle and a change of direction. At an angle more support may be required for the change of direction. Therefore, the closer to an angle the vertical support is the more support the footwear may provide. As a result, more cross foot support to the foot may be provided in the embodiment of FIGS. 6, 7 than in the embodiment of FIGS. 5, 5A which is closer to vector 19. As in the embodiment of FIG. 7 is farther forward on the lateral side towards the point of triangulation than FIG. 6 there may be more support and bracing in this footwear. FIG. 7 and similar footwear may also provide more cross foot support and bracing due to being closer and more approximately across from the first metatarsal, another point of triangulation.

The foot is a triangle shape with initial impact during a footstep to the rear foot point of triangulation. Muscles and tendons are often attached in the rear and mid foot for power to move the foot forward. The triangle is wider in front. This allows for lateral and medial movement and momentum across the forefoot. Part of human balance is achieved through the adjustments of lateral and medial muscle movements in the forefoot. Abductor muscles in the foot pull towards the lateral and adductor muscles pull toward the medial side. The foot in motion generally proceeds forward through a triangulated sequence and the invention systematically and methodically follows. The footwear may contain elements in the rear foot, on the lateral and the medial sides. As a result footwear manages the triangulation of the foot in motion. This occurs whether or not the footwear contains vertical elements at the angles of triangulation.

In the embodiments of FIGS. 6, 7 tension and compression is created within the embodiment at 21, 22 on the medial side. Similar tension and compression is created in areas lateral vertical beside cuboid bone 47, lateral horizontal below cuboid bone 48 in FIG. 6 and lateral vertical beside base of fifth metatarsal 50, lateral horizontal below base of fifth metatarsal 51 indicated by two headed arrow 49 on the lateral side in FIG. 7. In FIGS. 6, 7, two headed arrow 49 on the lateral side shows similar tension and compression to 27, on the medial side, in 21, 22 in FIGS. 4, 4A, 5, 5A.

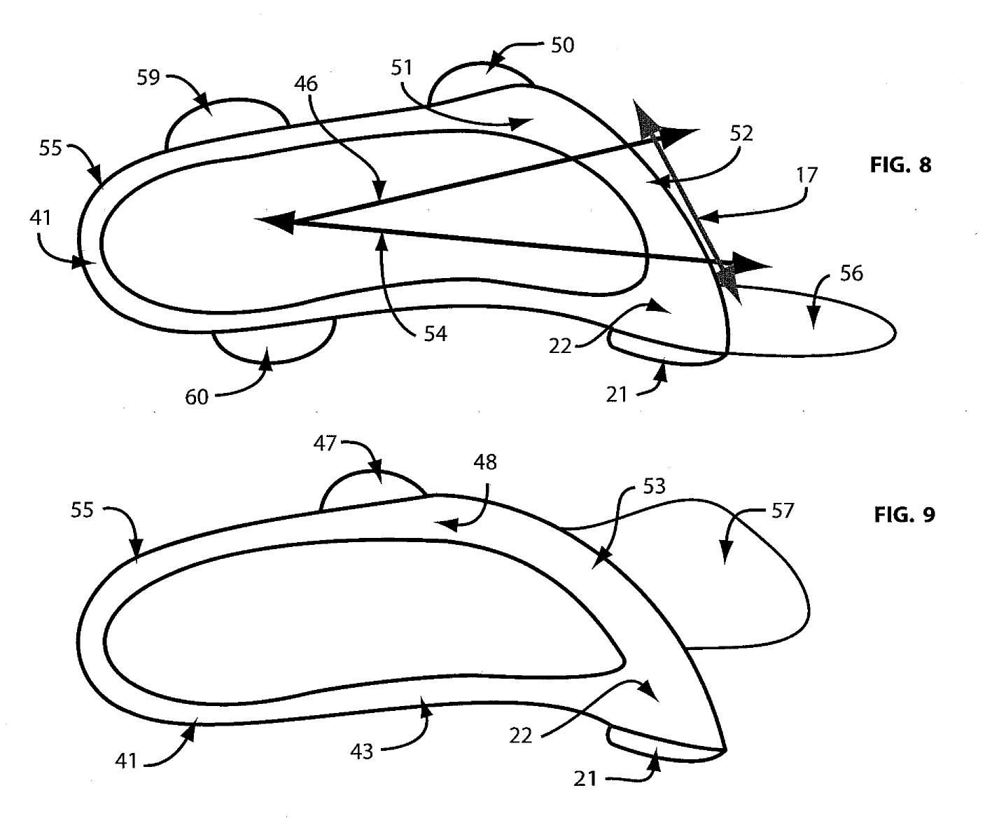

As in the tension and compression within 21, 22 in previously discussed footwear and 47, 48 and 50, 51 in embodiment FIGS. 6, 7, so too tension and compression is created within 21, 22 and 50, 51 in FIG. 8 and within 21, 22 and 47, 48 in FIG. 9. The upper of footwear in FIG. 8 includes 21; 50, 59, 60 and the sole includes 22, 51, 41, 55, 52. The upper of footwear in FIG. 9 includes 21, 47 and the sole includes 22, 48, 41, 43, 55, 53. All following footwear with areas 47, 48, 50, 51, 104, 105 on the lateral side and 21, 22 on the medial side are in tension and compression with the foot in motion.

The FIG. 8 embodiment contains cross foot bridge 52 along vector 17. The triangulated shape of the foot indicated by arrows 46, 54 and directional force vector 17 and the triangulated form of the embodiment FIG. 8, are somewhat above and below each other. The FIG. 8 embodiment more nearly imitates the connected triangulation of the three points of support 30, 31, 32 of the foot in motion due to the bridge 52 across the foot from the lateral to the medial side than previous embodiments. As an embodiment with portion 50 beside the rear base of the fifth metatarsal 31 and portion 21 beside the first metatarsal 32 and with a bridge underneath the foot 51, 52, 22, the FIG. 8 structure allows the more stationary base of the fifth metatarsal to interact with the more medial and lateral movements of the first metatarsal 32 during the forward motion of a foot.

The position of the rear portion of the fifth metatarsal 31 pressed up against the cuboid bone 40 and the fourth metatarsal 36, shown in FIG. 3C, limits its medial movement. The limited movement may be beneficial; however sports injuries occur there, possibly due to its limited movement. The portions 50, 51 may provide barriers beside and below the foot and support to foot movement. The first metatarsal 32 moves more both medially and laterally than the rear base portion of the fifth 31. Portion 50 contains momentum from medial to lateral vector 14 in FIG. 1. The limited medial mobility of the rear base of the fifth metatarsal holds vertical portion 50 of the embodiment relatively in place as directional force FIG. 1, 15 creates cross foot tension between horizontal lateral 51 and vertical medial 22. Horizontal lateral 51 is placed in tension as downward pressure is placed on it while directional force moves momentum to vertical medial 22. The tension in vertical medial 22, on the opposite side, pulls horizontal lateral 51 vertically against the downward pressure on it. This creates tension in the footwear that extends across the foot. This results in cross foot tension and compression in footwear. This cross foot tension absorbs shock, braces and supports the first metatarsal 32 of the foot. Tension in embodiments with a lateral vertical like 50, a bridge 52, or other lateral to medial bridges, and a medial vertical like 21 creates tension across footwear from the lateral to the medial side. Although FIG. 9 has a cross foot bridge 53 located more towards the rear foot vector 18 and away from the point of triangulation at the base of the fifth metatarsal it still has cross foot tension and compression. There is also cross foot tension and compression in footwear from the medial to the lateral side when medial to lateral vector 14 in FIGS. 1 and 17, 18 in FIG. 2 create tension in vertical laterals 50, 47 while horizontal medial bottom portion 22 is pulled vertically. This cross foot tension and compression is in addition to tension and compression within portions of the footwear on just the lateral 50, 51 and 47, 48 and medial 21, 22 sides for examples.

The embodiments of FIGS. 8, 9 are not attached to shoe counters 24. Portion 41 of embodiments FIGS. 8, 9 is a horizontal portion underneath the foot, and 55 indicates an outer line of the footwear in the rear foot that may or may not be beside the counter to stabilize the rear foot area of the footwear. Any embodiment of the invention may be separate from the counter or attached.

FIG. 8 also shows portions 59 on the lateral and 60 on the medial side of the rear foot. These are substantially horizontal portions extending to the counter of footwear to hold the rear foot area of this and other embodiments to footwear. Portions 59, 60 may provide another way to stabilize footwear by having a vertical portion proceeding up beside the sides of the rear foot. Vertical portions extending up beside the heel on the lateral and the medial side may stabilize the rear foot and rear foot portion of footwear thereby assisting in holding the rest of the embodiment in position to sustain the foot. In footwear vertical rear foot stabilizing portions 59 may be connected to vertical support 50 in FIG. 8 and to 47 in FIG. 9. Horizontal or horizontal and vertical rear foot stabilizing portions 59, 60 may be added singularly or both together to footwear without connections to the counter. They may be adjustable to the rear foot width and height of users and have differing configurations. Footwear may have vertical portions 59, 60 extending back and around the rear foot area of the embodiment and be joined there or not.

The embodiments of FIGS. 8, 9 show forefoot additions 56, 57. They may be customized to a specific consumer's foot concern. In FIG. 8, cushioning 56 is added to the embodiment at the area of the first metatarsal head and phalanges 34, 35. In FIG. 9 cushioning 57 is added to the embodiment at the second 38, third 37 and fourth 36 metatarsal heads and phalanges. Foam or similar material may be placed above and or below and or as part of embodiments of the footwear. When the footwear comprises a plastic or plastic like material that is harder, and/or denser than foam or rubber-like material, it can act as a shell to which other shoe features can be added, for example arch supports.

Current treatments for metatarsal pain may place a raised, cushioned pad just behind the second, third and fourth metatarsal heads, relieving some of the downward pressure on the metatarsal heads by moving it back towards the rear foot and spreading it over a wider area, lessening it at the metatarsal heads. As an alternative, a cushioned pad could be added to the bridges, 52 in FIG. 8 and 53 in FIG. 9. Further, as the material of the footwear can be molded, is rigid, yet is flexible, the shape of the bridges 52 and 53 in FIGS. 8, 9, and other footwear can be constructed to accommodate the cushioned forefoot pads. Also, if the embodiment is custom made, the bridge can be designed to accommodate the structure of the forefoot of the patient. Bridge 52 with 50, 51 beside the fifth metatarsal 31 is often used in further embodiments. It is understood bridge 52 and 53, beside and under the cuboid bone 40 portions 47, 48 can both be used in footwear.

The lateral to the medial side portions 52, 53 in FIGS. 8, 9 may benefit from utilizing bending portions 44A, 44B in FIG. 5. It is understood there are other ways to allow the material of the footwear to bend and other embodiments may benefit from bending portions 44A, 44B in FIG. 5.

Foot pressure and force are exerted through the bones to the joints. Excessive pressure or pressure in the wrong place or direction can affect the joints. A function of the footwear is to absorb momentum and possibly flex in portions 21, 22, 47, 48, 50, 51, 104, 105 in order to absorb shock and brace the non-bending bones of the forefoot to reduce the chances for injuries at the joints. The joints are often the areas of injury. They are where and when the maximum of downward pressure and directional force repeatedly occurs. The flexing of the material of the footwear contains directional force, momentum and force to the bones of the foot.

Sports requiring lateral, side to side, foot movement, like tennis, soccer and basketball may benefit from the physical triangulation of these forefoot bridges 52, 52A, 53 and other cross foot bridges. These bridges complete the connection of the three areas of support of the foot and mimic the natural triangulation of the foot. The mimicking triangulation of the footwear braces and stabilizes these angles when downward pressure and transition to other directions are at these angles of triangulation of the foot.

Cross foot tension and compression and triangulation may be managed by the footwear. By containing the force of lateral and medial momentum without appreciably limiting the extent of foot movement, athletes in sports that require frequent lateral and medial foot motion, may recover from their sideways movements more quickly.

By custom constructing the present footwear for athletes valuable information can be gained. Athletes concerned with directed forward motion, like marathon runners and sprinters may benefit. Long distance runners may experience muscle fatigue. They may benefit from the cumulative bracing effect on bones that may reduce stress on the muscles. Better athletic performance may result as the extremes of pressure and force at the lateral and medial sides of the foot may be lessened through containment in the footwear.

The material of the footwear can be calibrated to accommodate tension and compression and make it appropriate to the requirements of the foot in motion. The thickness of the material may vary according to its function. The plastic or plastic like material can also include a fibrous or other type of embedded reinforcing material, thereby creating a composite material that may, for example act directionally or cross directionally. Further, a mechanism can be combined with the footwear to test, for example the tensile strength and other elements of the footwear. This mechanism can provide data from the footwear to manufacturers and consumers. The information can be applied to footwear for the mass consuming public and those requiring the footwear for foot related problems. The material of footwear may be a version of existing foot orthotic material, shoe counter material, a new material, a material adapted for use with footwear, a material used for orthotics in other parts of the body and/or a combination. The material may have characteristics of flexible, semi-rigid and rigid foot orthotics. When choosing the strength of a material in any such category the amount of weight and forces on the material must be considered. The footwear may be a combination, in layers, in portions, thicknesses, flexibility and relative firmness of material. When weight is placed by the foot on the footwear it responds. As a result, weight does not have to be on the foot when custom making footwear. Slight weight on the molding material can produce a flat bottom to the footwear allowing it to have a flat bottom when placed in shoes. Heated or soaked material can be placed on the foot or a plaster foot cast. Alternatively, a three dimensional computer image of a foot may be made and corresponding footwear of the invention created. Carboplast™ is a brand of light weight durable material made of high strength carbon and glass fibers in a polypropylene matrix. The models are made from a sheet of Standard Carboplast material in a thickness of 2.5 mm. A 2.5 mm thickness is considered rigid. It can be heated in an oven to soften the material. The material as softened can be placed over a foot mold where it is cooled and hardened. Further fittings require trimming and adjustments to the footwear.

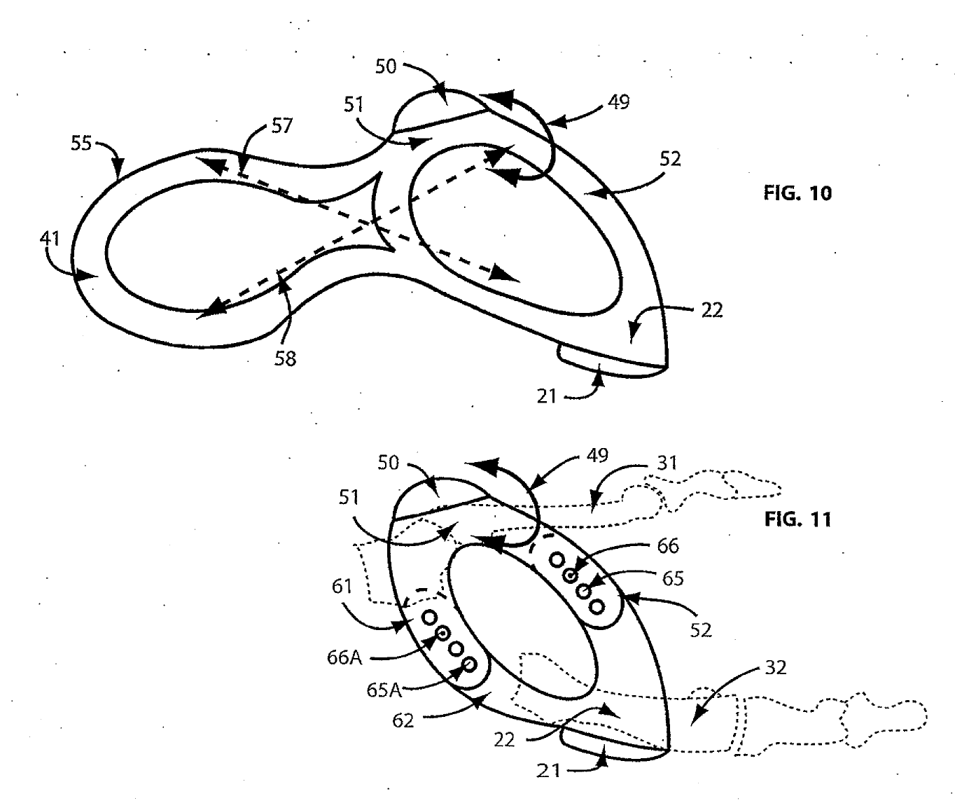

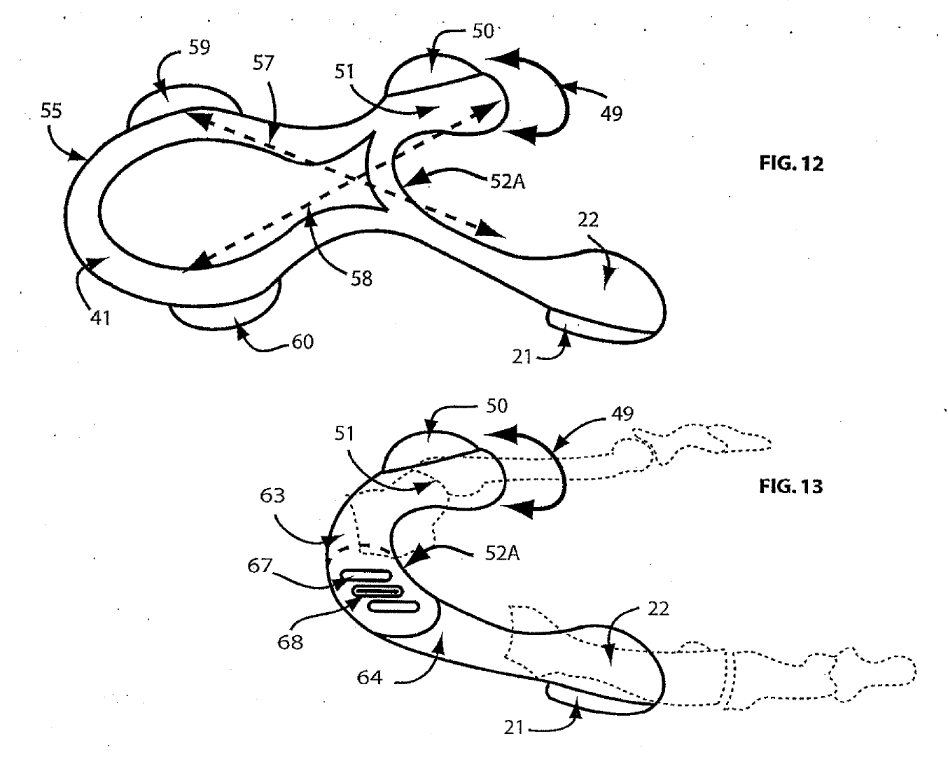

The embodiments of FIGS. 10, 12 comprise what may be called a “cross” or “X” shape. Two headed directional arrows vectors 57, 58 show directional forces crossing to opposite sides. Vector 57 crosses from lateral to medial and 58 from medial to lateral. By locating portions of embodiments in the middle under the foot the downward pressure from the rear foot along 57, 58 to opposing sides may increase lateral and medial stability in footwear and provide more support to the foot in motion. The upper of footwear in FIG. 10 includes 21, 50 and the sole 41, 55, 51, 22, 52. The upper of footwear in FIG. 12 includes 21, 50, 59, 60 and the sole 41, 55, 51, 22, 52A.

The embodiments of FIGS. 12 and 13 have bridge 52A further back towards the rear foot than bridge 52 in FIG. 10. The maximum bending of back to front foot motion in the short, fast races of sprinters may require more back to front flexibility than side to side. The location of bridge 52A, further back toward the rear foot where less foot bending occurs, may be of advantage to sprinters who require more back to front foot bending than other athletes require.

The embodiments of FIGS. 10, 11, 12, 13 do not show attachments to a counter 24, 41. FIG. 12 shows wings 59, 60 that may contact the counter 24, 41 to further stabilize the rear foot part of the footwear. It is understood there are many ways, including vertical portions, to implement these wings to further stabilize footwear. FIGS. 10, 11, 12, 13 portions 50, 51 are in tension, indicated by two headed arrow 49. When a foot is in forward motion, all footwear with lateral vertical side and lateral horizontal bottom portions may be in tension and compression similar to 49 in FIGS. 10, 11, 12, 13. Further, all footwear with portions 21, 22 may be in tension and compression. All footwear with portions extending from the lateral to medial side similar to 42, 52, 53, 52A, 112 may be in cross foot tension and compression and triangulation.

FIGS. 11, 13 each have two parts to adjust the width and length of footwear. FIG. 11 has parts 61, 62 with holes 65, 65A and plug 66, 66A. FIG. 13 has parts 63, 64 with slots 67 and plug 68. Other means of adjustment may be used and are still within the scope of the present invention. The upper of footwear FIGS. 11, 13 includes 21, 50, while the remainder is in the sole. Downward compression at the rear foot begins triangulation, as a result FIGS. 11, 13 and others with lateral and medial elements triangulate.

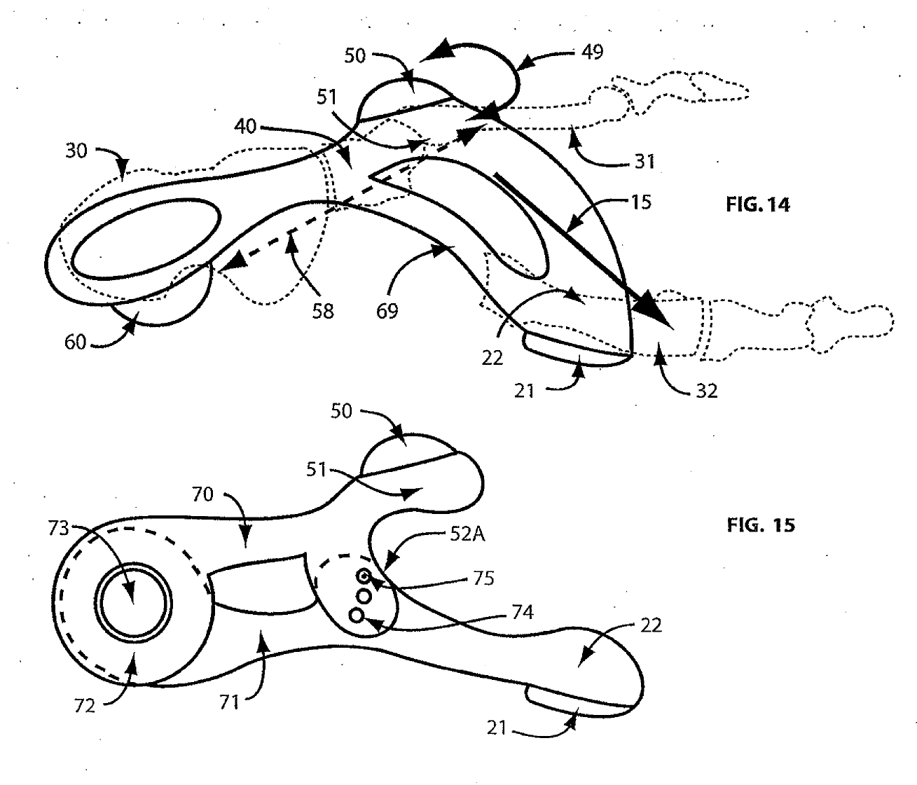

The embodiment of FIG. 14 has two headed vector arrow 58 in dashed lines proceeding from the rear foot medial side to the forefoot lateral side and vector arrow 15 from the lateral to the medial side. Arrows 58 and 15 indicate the movement of downward pressure and directional force and somewhat mirror the embodiment of FIG. 14 and the placement of some of the bones of the foot, indicated in smaller dashed lines, including calaneus 30, cuboid 40, fifth 31 and first metatarsal 32. Embodiment FIG. 14 also shows portion 60 which may have horizontal and vertical portions in the area beside the rear foot on the medial side. Vertical and horizontal portions 60, 50, 51, 21, 22 create the shape of a triangle with two portions on the medial and one on the lateral. This triangulation may stabilize footwear. The footwear may triangulate with two angles on the lateral, rather than one as in FIG. 14, and one on the medial, rather than two. FIG. 14 also shows additional optional bridge 69 from the lateral to the medial side. The upper of footwear includes 21, 50, 60, while the remainder is in the sole.

The embodiment of FIG. 15 has two pieces 70, 71. Part 70 has outer circle 72 to swivel for width adjustment and an array of holes 74 for footwear. Part 71 has inner circle 73 below 70 and plug 75 below 70 that fits into any of the holes 74 to hold the width adjustment of footwear in place. The upper of footwear includes 21, 50, while the remainder is in the sole.

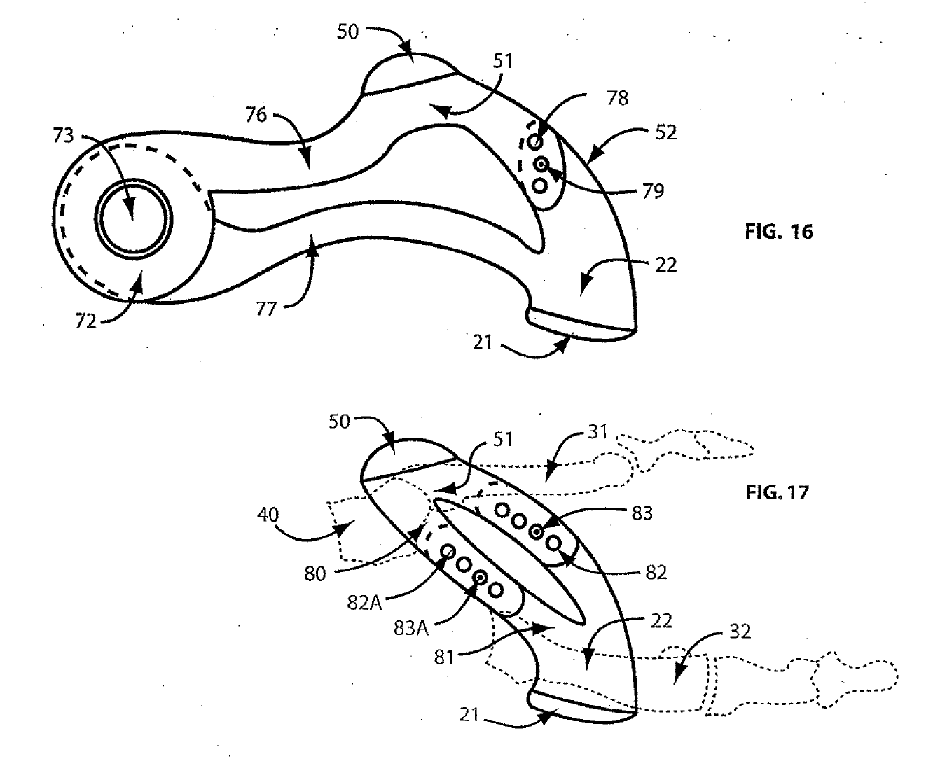

The embodiment of FIG. 16 has two pieces 76, 77. Part 76 has outer circle 72 to swivel for width adjustment and an array of holes 78. Part 77 has inner circle 73 below 76 and plug 79 below 76 that fits into any of the holes 78 to hold the width adjustment of footwear in place. The upper of footwear includes 21, 50, while the remainder is in the sole.

The embodiment of FIG. 17 has two pieces 80, 81. Part 80 has holes 82 forward and 82A rearward and part 81 has plug 83 forward and 83A rearward for width adjustment of footwear. Cuboid bone 40, fifth metatarsal 31 and first metatarsal 32 are shown in dashed lines. The upper of footwear includes 21, 50, while the remainder is in the sole.

Foot health may improve due to limiting extension of the foot by the structure and material of the invention during rigorous athletic and normal activity. Containing the lateral and medial extension may increase the possibility of a repetitive, stable, foot-strike. If the footwear of the invention is inserted in different shoes by the consumer it achieves a consistency of forward foot movement relatively independent of the choice of shoes. As mentioned above, it is often suspected a relatively repetitive, stable foot-strike that is not painful is advantageous.

Metatarsalgia is a general term for pain in the area of the forefoot metatarsal heads. Some of its causes are: shoes that squeeze the toes, athletic activities that apply great pressure on the foot and the diminishing of the fat pad under the metatarsal heads as we age. A hypermobile first metatarsal is a foot problem. Morton's Neuroma is a forefoot condition occurring between the third and fourth metatarsal heads. Morton's Toe can occur if the second toe is longer than the first. This is possibly due to the thinner second metatarsal head being longer than the first and absorbing pressures that would otherwise be absorbed by the thicker first metatarsal head if it was longer instead. These and other problems at or near the metatarsal heads and forefoot may benefit from use of the invention.

Bunions, medically know as Hallux Valgus at the first, and bunionettes at the fifth metatarsals are a foot problem often linked to women's high heel shoes. The pain of bunions may be mitigated by footwear of the invention. During a normal footstep there is an exchange of force and pressure from the rear to the forefoot and from the lateral to medial side. High heel shoes restrict the toes from the natural moving forward and spreading movement that absorbs the forward pressure and directed force of the foot in motion. With each step in high heel shoes the directed pressure limits the forward movement and spreading of the toes. The toebox is the area of a shoe that contains the toes of the foot. The toebox may restrict the natural forward movement and spreading of the toes in order to keep the rear of the foot from sliding down off the top of the high heel and the forefoot from poking out the front of the footwear. Further, the cramped toebox of a shoe is often pointed forcing the toes towards the center of the foot rather than allowing the natural spreading of the toes to the sides. The forced movement of the toes towards the center of the foot and the limiting of their forward motion is the opposite motion of the toes natural tendency to spread and move forward. As a result, the steep angle of the rear foot in high heels places inordinate amounts of downward pressure repeatedly on the same forefoot metatarsal heads rather than disbursing it forward. This rear downward pressure also forces the metatarsal heads to spread. At the same time, opposing directional force from the toes in the cramped toebox is applied back to the metatarsal heads, further forcing the metatarsal heads to spread apart from each other. The overwhelming force from the first and fifth metatarsals behind the toes extends them farther to the sides of the foot and forces the toes in front of them further into the cramped toebox. During a footstep and over time, use of high heel shoes spreads metatarsals more, both laterally and medially, than would a flatter soled shoe with a wider and longer area in the forefoot toebox. Eventually the metatarsals may become permanently spread causing foot pain.

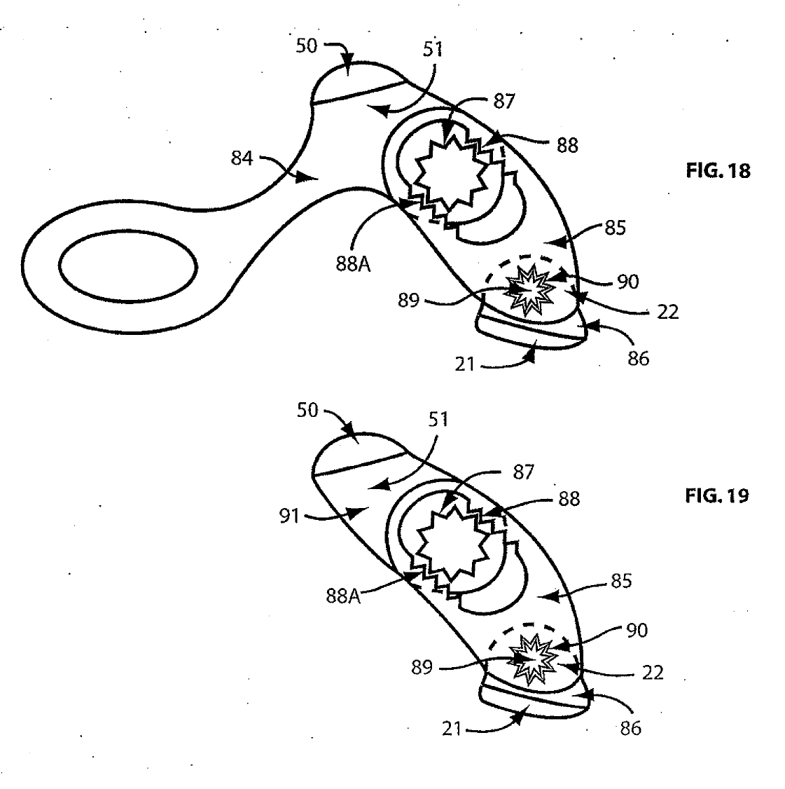

Bracing and support of the first and fifth metatarsals by footwear of the invention may mitigate the pain of bunions and other foot conditions. The width and spreading angles of the first and fifth metatarsals from wearing high heel shoes are different for different people and foot sizes. As a result, several of the embodiments following have inner spiked wheels 89 and outer spiked wheel 90 on the medial and/or lateral sides, as shown in FIGS. 18, 19, 20, 21, 22, 23, 28 and 29. Spiked wheels 89, 90 allow angle portions 50, 51, 104, 105 and 47, 48, on the lateral side and 21, 22 on the medial side to adjust to different angles of the forefoot metatarsals on the lateral and medial sides of footwear.

It is understood spiked wheels 89, 90 can be added to footwear mentioned above as well. It is understood other footwear may include spiked wheels 89, 90 on the lateral or medial side only or on both sides. It is also understood there are other means of allowing footwear to adapt to angles of the metatarsals. It is also understood a swivel mechanism like 72, 73 in the rear foot may be applied to the forefoot in order to self adjust to metatarsal angles during forward motion of the foot in footwear.

The embodiment of FIG. 18 has three pieces 84, 85, 86. Part 84 has a rear foot portion to help stabilize the embodiment. It is understood the rear foot portion of the footwear can take on a different shape. It is also understood that the footwear can effect triangulation with or without a vertical upper portion. Part 84 has a circular shape portion with teeth 87 that fits under teeth portions 88, 88A on both sides of 85 to adjust the width of footwear. FIG. 18 includes circular portion 87 that can also adjust to brace and support different portions along the length of the first metatarsal bone if placed at different angles on in line teeth 88, 88A on both sides of 85 of footwear. Part 85 also has circular portion 90 that can be placed over portion 89 of part 86. By placing 89 of 86 under 90 of 85 at different angles footwear can be aligned to the first metatarsal bone. The upper of footwear includes 21, 50, while the remainder is in the sole.

The embodiment of FIG. 19 has three pieces 91, 85, 86. Part 91 has a circular shape portion with teeth 87 that fits under teeth portions 88, 88A on both sides of 85 to adjust the width of footwear. FIG. 19 includes circular portion 87 that can also adjust to brace and support different portions along the length of the first metatarsal bone if placed at different angles on in line teeth 88, 88A on both sides of 85 of footwear. Part 85 also has circular portion 90 that can be placed over portion 89 of part 86. By placing 89 of 86 under 90 of 85 at different angles the footwear can be aligned to the first metatarsal. The upper of footwear includes 21, 50, while the remainder is in the sole.

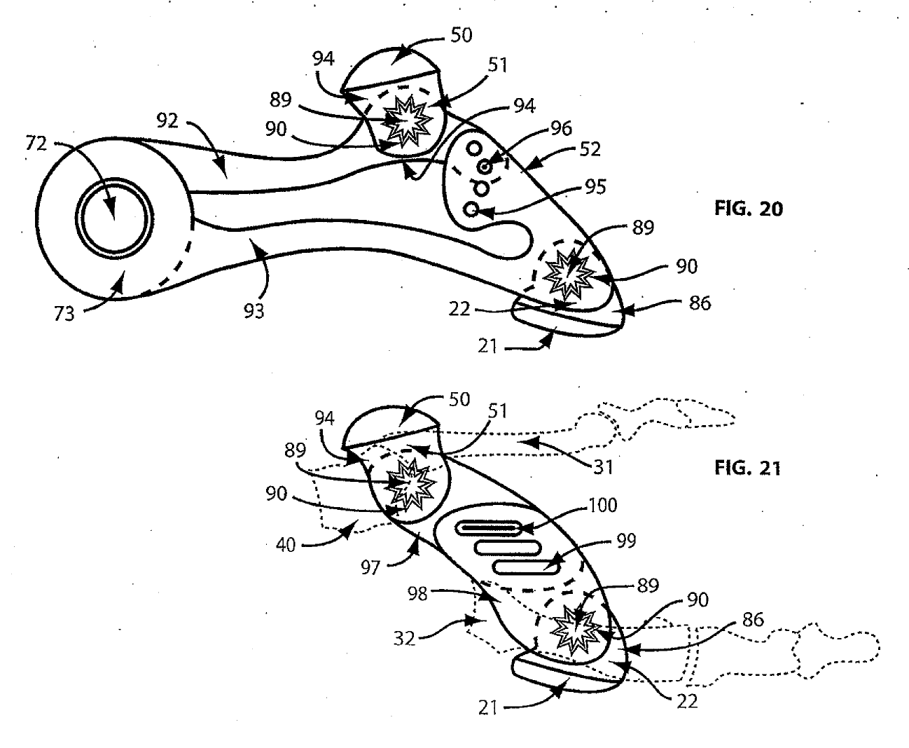

The embodiment of FIG. 20 has four pieces 92, 93, 86, 94. Piece 92 has inside circular portion 72 to allow the piece to swivel in conjunction with piece 93 outside circular portion 73 of footwear. Piece 92 has inside teeth portion 89 fitting under outside teeth portions 90 of piece 94 to adjust to the angle of the fifth metatarsal of footwear. Piece 93 has outside teeth portion 90 fitting over inside teeth portions 89 of piece 86 to adjust to the angle of the first metatarsal of footwear. The array of holes 95 of piece 93 allows plug 96 of 92 to enter under to adjust the width of footwear. The upper of footwear includes 21, 50, while the remainder is in the sole.

The embodiment of FIG. 21 has four pieces 97, 98, 86, 94. Piece 97 has inside teeth portion 89 fitting under outside teeth portions 90 of piece 94 to adjust to the angle of the fifth metatarsal of footwear. Piece 98 has outside teeth portion 90 fitting over inside teeth portions 89 of piece 86 to adjust to the angle of the first metatarsal of footwear. Slots 99 of piece 98 allows plug 100 of 97 to enter underneath to adjust the width of footwear. Some bones of the foot are shown in dashed lines. The upper of footwear includes 21, 50, while the remainder is in the sole.

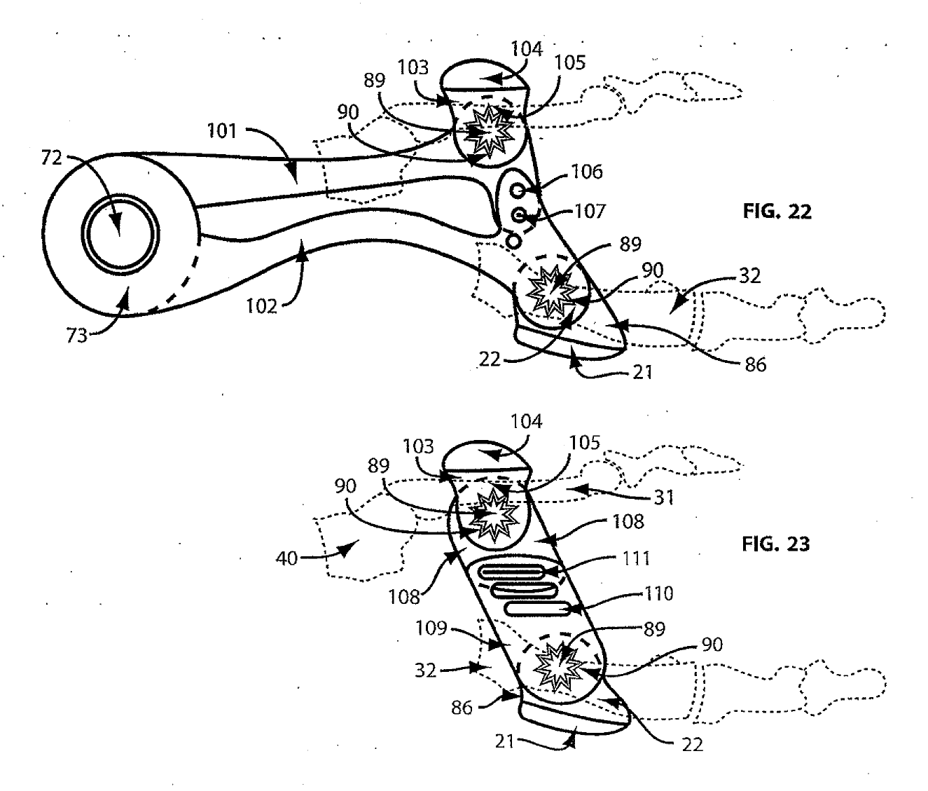

The embodiment of FIG. 22 has four pieces 101, 102, 103, 86. Piece 101 has inside rear foot area circular portion 72 to allow the piece to swivel in conjunction with piece 102 outside circular rear foot area portion 73 of footwear. Piece 101 has inside teeth portion 89 fitting under outside teeth portions 90 of piece 103 to adjust to the angle beside and further forward than the base of the fifth metatarsal. Piece 102 has outside teeth portion 90 fitting over inside teeth portions 89 of piece 86 to adjust to the angle of the first metatarsal. The array of holes 106 of piece 102 allows plug 107 of 101 to enter under to adjust the width of footwear. FIG. 22 has piece 103 with portions 104, 105 providing similar tension and compression in footwear in a different location at the fifth metatarsal 31 than 50, 51. All footwear with areas 47, 48, 50, 51, 104, 105 on the lateral side and 21, 22 on the medial side are in tension and compression with the foot in motion. Some bones of the foot are shown in dashed lines. The upper of footwear includes 21, 104, while the remainder is in the sole.

The embodiment of FIG. 23 has four pieces 108, 109, 103, 86. Piece 108 has inside teeth portion 89 fitting under outside teeth portions 90 of piece 103 to adjust to the angle beside and further forward than the base of the fifth metatarsal of footwear. Piece 109 has outside teeth portion 90 fitting over inside teeth portions 89 of piece 86 to adjust to the angle of the first metatarsal. Slots 110 of piece 109 allow plug 111 of 108 to fit underneath to adjust the width of footwear. Some bones of the foot are shown in dashed lines. The upper of footwear includes 21, 104, while the remainder is in the sole.

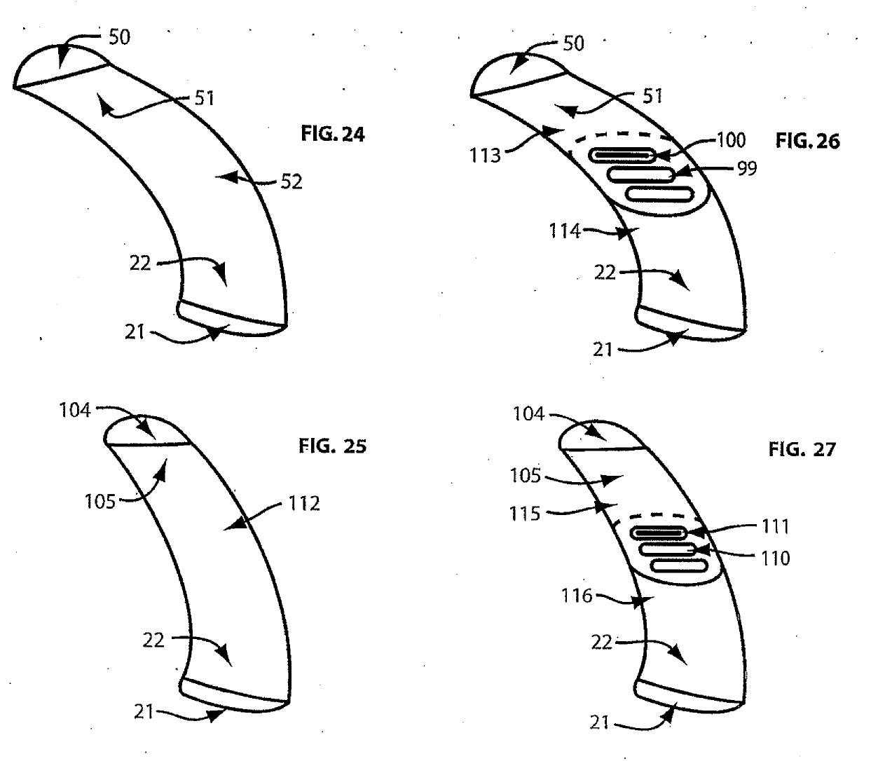

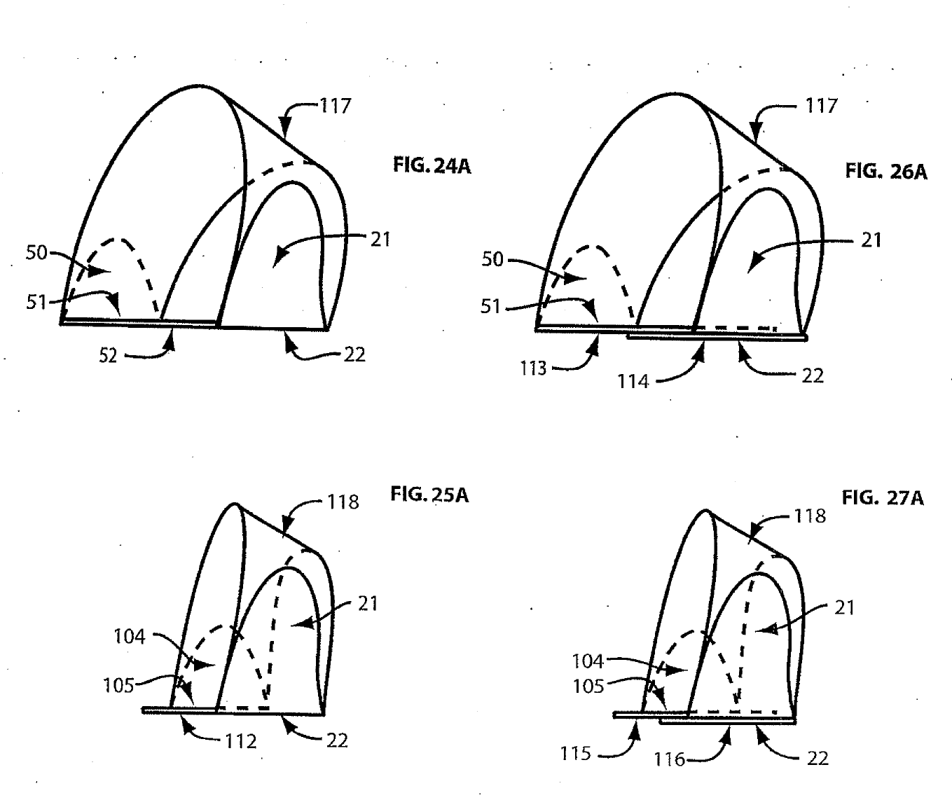

The embodiment of FIG. 24 is one piece with portions 52, 21, 22, 50, 51. FIG. 26 is similar to FIG. 24, but with two pieces 113, 114 with portions 21, 22, 50, 51, 99, 100. Slots 99 of 113 allow plug 100 of 114 to fit underneath to adjust the width of footwear. The upper of footwear includes 21, 50, while the remainder is in the sole.

The embodiment of FIG. 25 is one piece with portions 112, 21, 22, 104, 105. FIG. 27 is similar to FIG. 25, but with two pieces 115, 116 with portions 21, 22, 104, 105, 110, 111. Slots 110 of 115 allow plug 111 of piece 116 to fit underneath to adjust the width of footwear. The upper of footwear includes 21, 104, while the remainder is in the sole.

In embodiments FIGS. 24A, 25A, 26A, 27A attachments 117, 118 are attached to the inner side of the lateral 50, 51, 104, 105 and medial 21, 22 sides. It is understood they can be attached to the outer side as well in these and other footwear. Embodiment FIG. 24A is a single piece 21, 22, 50, 51, 52 with attachment 117. Embodiment FIG. 25A is a single piece 21, 22, 104, 105, 112 with attachment 118. Single piece footwear like embodiments FIGS. 24, 25, 24A, 25A may be footwear manufactured in different sizes, custom made to the foot width of consumers and patients and customizable by consumers. FIG. 26A has two pieces 113, 114 with slots 99 and plug 100 and FIG. 27A has two pieces 115, 116 with slots 110, and plug 111. FIGS. 26A, 27A have attachments 117, 118 respectively. Embodiments FIGS. 26, 27, 26A, 27A and footwear similar to these may accommodate a range of width sizes.

Portions 117, 118 are in place over the top of the foot and attached on the medial 21, 22 and lateral sides 50, 51, 104, 105 of the embodiments. It is understood 117, 118 may continue under the footwear as well. It is understood an adjustable mechanism like 117, 118 over the top of the foot may not be attached to FIGS. 24A, 25A, 26A, 27A or other footwear and may be a separate entity and mechanism. It is also understood a separate entity with portions 117, 118 can also have additional portions under the foot. It is also understood existing devices that wrap entirely around the circumference of the foot with portions like 117, 118 and portions under the foot can be used with, attached to, overlap, or placed within footwear of the invention. It is also understood the mechanism tightening over the top of the foot can also be laces or other closure devices, such as shoelaces on the outside of shoes. It is also understood footwear of the invention with or without attachments like 117, 118 can be the featured aspect of the invention's design, manufacture and marketing.

The material of 117, 118 can be stretchable elastic, a material with laces or another type of adjustable fastener. It can adjust to the required tightness or looseness over the top of the foot and assist in holding portions of the footwear beside and under the foot in position. However the tightness or looseness of 117, 118 and similar mechanisms mentioned above are relatively independent of the tightness or looseness of the remainder 21, 22, 50, 51, 52, 104, 105, 112, 113, 114, 115, 116 of the footwear and embodiments in FIGS. 24A, 25A, 26A, 27A and other embodiments. Therefore the tightness or looseness on the lateral and medial sides of the foot can be adjusted separately from the tightness or looseness over the top of the foot.

In alternate embodiments, the ability to adjust the tightness or looseness on the lateral and medial sides of the foot relatively independent of the tightness or looseness over the top of the foot allows the wearer to utilize the loosening and tightening capabilities of the footwear with or without attachments 117, 118 and or similar mechanism.

It is understood many embodiments of the invention may be combined by the manufacturer or the consumer with an entity and or mechanism that goes over the top of the foot similar to 117, 118 and also may continue underneath the foot wrapping the circumference of the forefoot in footwear.

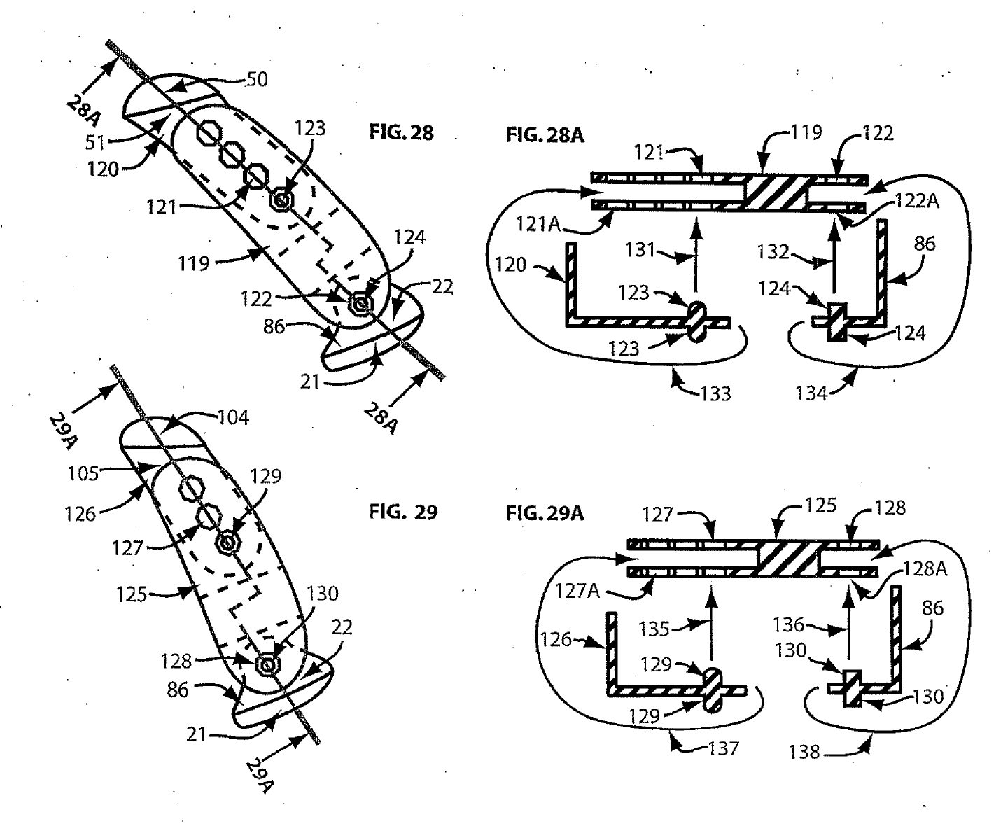

The embodiment of FIG. 28 has three pieces 119, 120, 86. Piece 119 has upper and lower holes 121, 121A that plug 123 of piece 120 can fit into to adjust the width of FIG. 28 and upper and lower holes 122, 122A that plug 124 of 86 can fit into to adjust to the angle of the first metatarsal of footwear. Piece 120 has portions 50, 51 and 123 to adjust to the angle of the fifth metatarsal 31. Piece 86 has portions 21, 22 and 124 to adjust to the angle of the first metatarsal 32 of footwear. Portions 121, 121A, 122, 122A, 123, 124 are polygons allowing them to adjust to angles. It is understood that in footwear there can be other configurations that can allow them to adjust to angles. In footwear portions 121, 121A, 122, 122A, 123, 124 may be circular to change angles as the angles of the first and fifth metatarsal bones change angles during forward movement. The upper of footwear includes 21, 50, while the remainder is in the sole.

The embodiment of FIG. 29 has three pieces 125, 126, 86. Piece 125 has upper and lower holes 127, 127A that plug 129 of piece 126 can fit into to adjust the width of FIG. 29 and upper and lower holes 128, 128A that plug 130 can fit into to adjust to the angle of the first metatarsal of footwear. Piece 126 has portions 104, 105 and 129 to adjust to the angle of the fifth metatarsal 31. Piece 86 has portions 21, 22 and plug 130 to adjust to the angle of the first metatarsal 32. Portions 127, 127A, 128, 128A, 129, 130 are polygons allowing them to adjust to angles. It is understood that in footwear there can be other configurations that can allow them to adjust to angles. Portions 127, 127A, 128, 128A, 129, 130 can be circular to change angles as the angles of the first and fifth metatarsal bones change angles during forward movement. The upper of footwear includes 21, 104, while the remainder is in the sole.

The embodiment of FIG. 28A is a cutaway view of FIG. 28 with the three pieces 119, 120, 86 separated. Arrows 131, 132 point toward holes 121, 121A, 122, 122A into which plugs 123, 124 respectively fit. Arrow 133 shows a possible way of inserting piece 120 into piece 119. In footwear it is understood plug 123 of piece 120 has a choice of holes 121, 121A in piece 119 to adjust the width of the embodiment FIGS. 28, 28A. Plug 123 of piece 120 has a rounded top and bottom portion to allow plug 123 to fit easily into holes 121, 121 A of piece 119. Plug 124 of piece 86 has a flat top and bottom portion. Arrow 134 shows a possible way of inserting piece 86 into piece 119. Plug 124 may be inserted in holes 122, 122A by the manufacturer. It is understood in footwear similar multiple holes 121, 121A and plug 123 on the lateral side may be on the medial side in embodiments. It is understood in footwear similar single holes 122, 122A and plug 124 on the medial side may be on the lateral side in embodiments.

The embodiment of FIG. 29A is a cutaway view of FIG. 29 with the three pieces 125, 126, 86 separated. Arrows 135, 136 point toward holes 127, 127A 128, 128A into which plugs 129, 130 respectively fit. Arrow 137 shows a possible way of inserting piece 126 into piece 125. It is understood in footwear plug 129 of piece 126 has a choice of holes 127, 127A in piece 125 to adjust the width of the embodiment FIGS. 29, 29A. Plug 129 of piece 126 has a rounded top and bottom portion to allow plug 129 to fit easily into holes 127, 127A of piece 125. Plug 130 of piece 86 has a flat top and bottom portion. Plug 130 may be inserted in holes 128, 128A of footwear by the manufacturer. Arrow 138 shows a possible way of inserting piece 86 into piece 125. It is understood in footwear similar multiple holes 127, 127A on the lateral side may be on the medial side in embodiments. It is understood in footwear similar single holes 128, 128A and plug 130 on the medial side may be on the lateral side in embodiments.

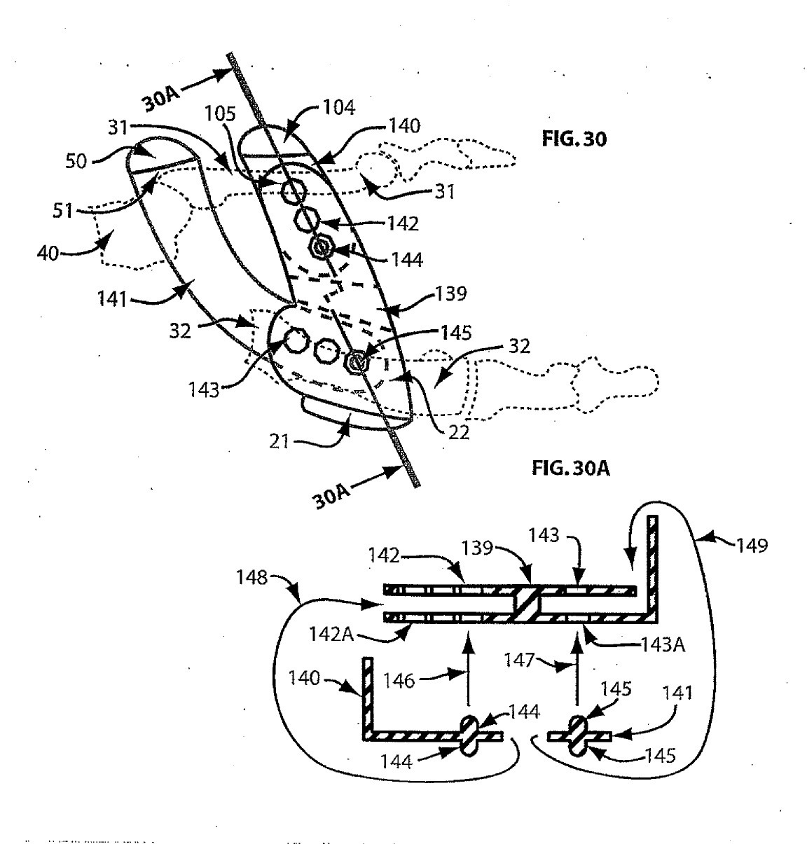

The embodiment of FIG. 30 has three pieces 139, 140, 141. Piece 139 has upper and lower holes 142, 142A that plug 144 of piece 140 can fit into to adjust the width of FIG. 30 at the middle of the fifth metatarsal. FIG. 30 has upper and lower holes 143, 143A of piece 139 that plug 145 of piece 141 can fit into to adjust to the width of FIG. 30 between the base of the fifth metatarsal and the first metatarsal 32. Piece 140 has portions 104, 105 and plug 144 to adjust to the angle of the fifth metatarsal 31 forward of its rear base. Portions 142, 142A 143, 143A 144, 145 are polygons allowing them to adjust to angles. It is understood in footwear there can be other configurations that can allow them to adjust to angles. Portions 142, 142A 143, 143A 144, 145 can be circular to change angles as the angles of the first and fifth metatarsal bones change angles during forward movement. FIG. 30 shows a cross foot triangulation between part 141, 140 on the lateral side and 139 on the medial. This triangulation may stabilize the embodiment. The stabilization may make footwear more effective. Sports and overuse injuries occur all along the fifth metatarsal. Footwear may benefit athletes and others by providing support there before or after an injury. Portions of footwear beside and/or below the fifth metatarsal may be elongated towards or away from each other similar to the upside down “L” shape in paragraph [0027]. This footwear may have additional portions, including towards the rear, to further stabilize it. The upper of footwear includes 21, 50, 104, while the remainder is in the sole.

The embodiment of FIG. 30A is a cutaway view of FIG. 30 with the three pieces 139, 140, 141 separated. Arrows 146, 147 point toward holes 142, 142A 143, 143A into which plugs 144, 145 respectively fit. Arrows 148, 149 show a possible way of inserting pieces 140, 141 respectively into piece 139. It is understood plug 144 of piece 140 has a choice of holes 142, 142A in piece 139 to adjust the width of the embodiments of FIGS. 30, 30A. Plug 144 of piece 140 has a rounded top and bottom portion to allow plug 144 to fit easily into holes 142, 142A of piece 140. Plug 145 of piece 141 has a rounded top and bottom portion to allow plug 145 to fit easily into holes 143, 143A of piece 139.

The following embodiments select the rear and forefoot and arch for structural features in footwear made from in plastic and plastic like material, rather than covering over the whole shoe sole with cushioning foam and or cushioning plastic.

The rear foot and forefoot are often areas in need of structures and of foot discomfort. The rear foot receives the greatest impact. The forefoot receives a change of direction of the foot and maximum flexing of the foot, combined with the thinnest bones, resulting in added stress to the joints there. Therefore the following embodiments rely on shock absorption for these areas of footwear, while cushioning can adequately serve the other areas.

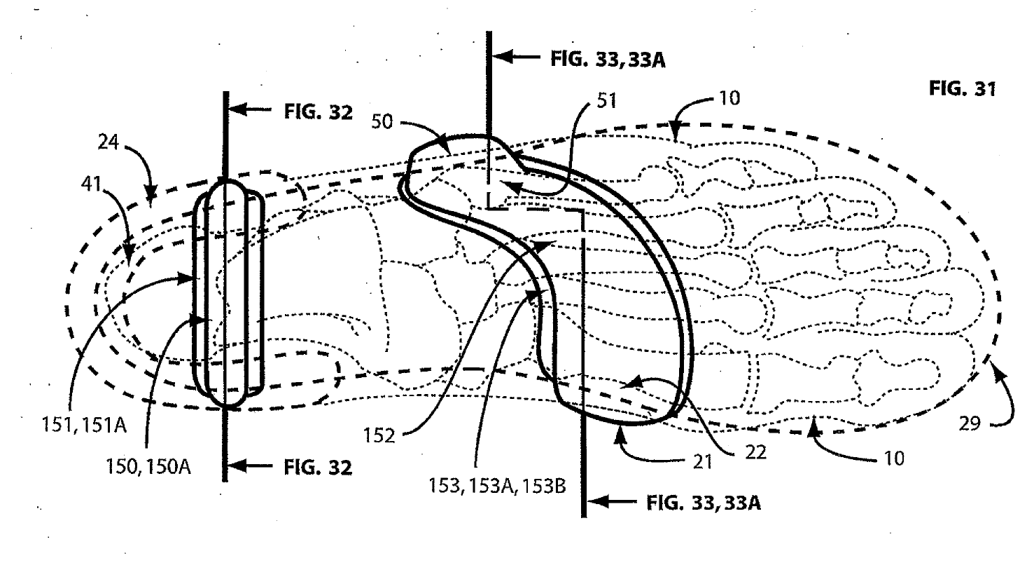

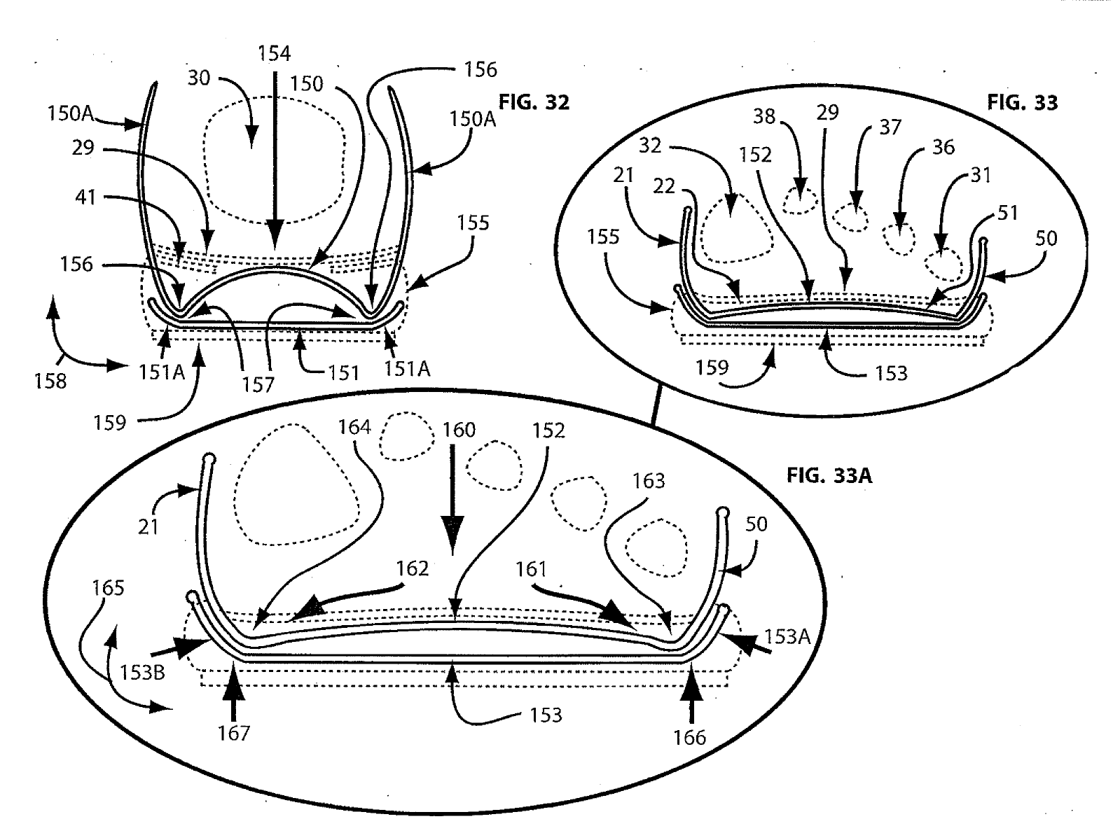

In a form of athletic shoe construction the completed upper is in an injection mold cavity where foam is shot into it to create the shape of the shoe sole and adhere it to the upper. If the footwear is a unit located in the upper and/or in the sole in other embodiments as well, as in FIG. 31 and cutaways FIG. 32, FIG. 33, FIG. 33A, the injection mold material can be formed around it in the shoe sole.

FIG. 31 shows a top view of footwear with cutaways FIGS. 32, 33, 33A. Shown in dashed lines are an outline of the foot 10 and an insole 29, an underneath portion of the counter 41 and an above portion 24. Bones of the foot are in thinner and shorter dashed lines. FIG. 31 shows portion 150, 150A above portion 151, 151A of FIG. 32. FIGS. 33, 33A are shown in FIG. 31 with portion 152, 21, 22, 50, 51 on top of portion 153, 153A, 153B.

In the embodiment of FIG. 32 plastic or plastic like material and/or metal is shown across sides of the rear foot with a convex portion 150 and a concave strike plate 151 that acts as a runner for the sides of convex portion 150 of footwear. The downward pressure 154 on the convex portion of 150 by foot impact almost simultaneously reacts back up toward the counter area and 150A of 150 of footwear. This may stabilize the ankle by pressing the counter upwards and inward towards the ankle and the calcaneus 30. The strike plate 151 utilizes the impact of the downward pressure 154 on 150 to deflect and thereby circulate the downward pressure over to the sides and back upwards to the portion 150A that may be connected to the counter 24. Resistance to downward pressure 154 is provided by 151, 151A to 150 and 150A, which may absorb some of the impact on the calcaneus 30. The absorption of impact by the footwear is compatible with the cushioning provided by the often used midsole foam material 155, indicated in dashed lines.

Portion 150 flexes downward, spreading and transferring the downward pressure 154 down its sides to the corners on both the lateral and the medial sides 156. Convex shape 150 diminishes in height and spreads sideways transferring sideways directional force into 151 and 151A. Portions 151, 151A of the embodiment are then in tension and resists the downward pressure across the strike plate 151 and at its corners 157 which remain rounded on the medial and lateral sides 151 A. The overwhelming resistance of portions 151, 151 A forces the rounded corners on the lateral and medial sides 156 of 150 to press against 151A of 151 and move up both sides of 151A. The downward pressure 154 is transferred into 150 then 151, then to 151A. The resistance of 151A of portion 151 allows portion 150A of 150 to rock up and back with the changes in downward pressure, indicated by two headed arrow 158. Areas 151A may require additional support and or shims.

The redirection of the downward pressure 154 in footwear back up the sides 150A may support the calcaneus 30 and the ankle during forward motion. This is a process of transfer of downward pressure to sideways directional force creating tension and directional force back upwards in footwear. Tension is in 150, 150A. It can be a circular motion that ends by bracing the rear of the foot and begins again with the impact of the rear of the foot again striking the ground plane of footwear.

With its attachment to both the upper and the sole, its integration of the differences between foot and footwear and its bio-mechanical utility, the footwear may become the organizing structure around which footwear is manufactured. For example, portion 150 of the embodiment combined with side portions 150A can be part of a shoe counter portion and part of the shoe sole, with portion 151, 151A as well.

Other footwear may include the convex fog n 150 with corners 156 without its upward facing sides 150A, but with the striking plate 151 with its upward facing sides 151 A. In this footwear, downward pressure 154 forces corners 156 to strike the lateral and medial sides of 151A. When the downward pressure 154 is removed the sides of the convex form 150 rock back 158 towards the center and the convex form 150 returns to its resting height and position above strike plate portion 151.

Other footwear may include a convex form 150 and strike plate platform 151 without sides 151A.

In other footwear portions 150, 151 without 150A, are joined on both sides respectively at 156 and the topmost points of 151A. When downward pressure is placed at the apex of 150 this form flattens somewhat at the apex distributing downward pressure to the sides. This relieves some pressure to the calcaneus 30 directly above the apex of 150 and moves it to the more pliable flesh at the sides of the heel. Heel spurs may occur at the heel due to extremes of downward pressure at foot to ground impact. By moving the downward pressure to the sides and the more pliable flesh heel spurs may be less likely.

FIGS. 33, 33A are cutaway views of the forefoot area of a footwear and foot shown in FIG. 31. The bones of the foot shown in dashed lines are the fifth metatarsal 31, first metatarsal 32, fourth metatarsal 36, third metatarsal 37, and second metatarsal 38. Dashed lines also show the midsole 155, which may be foam or plastic, the bottom sole 159. FIGS. 33, 33A solid lines include the embodiment portion 152, 21, 22, 50, 51 and portion 153 with 153A on the lateral side and 153B on the medial.

FIG. 33A is a larger cutaway view version of FIG. 33 of the forefoot area of footwear and forefoot bones indicated in FIG. 31. FIG. 33A indicates downward pressure 160 from the forefoot onto portion 152 of the embodiment. The downward pressure is transferred from portion 152 along vector arrows 161 on the lateral and 162 on the medial sides of footwear. Portion 152 flexes downward transferring the downward pressure down its sides to the corners on both the lateral 163 and the medial 164 sides. It is understood these corners 163, 164 can be curved to allow a rocking motion 165 of the corners portions of 152 on the lateral and medial sides of footwear.

Portion 153 of FIG. 33A contains rounded portions 166 on the lateral and 167 on the medial side of 153. Portion 153 of the embodiment resists the downward pressure from 152 at its corners on the lateral 166 and medial 167 sides. The overwhelming resistance of these corners of portion 153 forces the rounded corners on the lateral 163 and medial sides 164 of 152 against and up both sides, 153A on the lateral, and 153B on the medial of 153. Portion 163 of 152 rocks up and back 165 along the lateral side of 153A, and 164 of 152 rocks up and back 165 along the media side of 153B of 153 of footwear. As a result, 50, 22 may move upward and curl inward providing an opposing force to the directional forces 15 in FIGS. 1 and 17, 18 in FIG. 2, during a footstep. This opposing force may increase tension and add additional support and bracing to the forefoot area of the footwear.

The amount of rocking motion 165, in the structures of FIGS. 32, 33, 33A can be calibrated by the shape and strength of the material of footwear in relation to the downward pressure. Downward pressure then sideways and then back up the sides with the rocking motion 165 dissipates and absorbs shock to the foot in motion. In embodiment 33, 33A this dissipation and shock absorption to the foot in motion occurs in addition to the dissipation and shock achieved in forefoot areas of previous embodiments.

Other footwear structure in the forefoot area is achieved by the inclusion of convex form 152 without its upward facing sides 21, 50 and with the striking plate 152 with its upward facing sides 153A, 153B. In this footwear portion 152 strikes the platform 153 with downward pressure, flexes downward and slides out towards the sides where momentum is absorbed by 153A 153B. When the downward pressure is removed the sides of the convex form 152 slide back towards the center of footwear and the convex form 152 returns to its resting height and position above portion 153.

Other footwear may include a convex form 152 and strike plate platform 153 without sides 153A 153B.

In other footwear portions 152, 153 are joined on both sides without 21, 50, 153A, 153B. Corners 163 on the lateral and 164 on the medial are joined to both ends of 153 that no longer has portions 153A, 153B. When downward pressure is placed at the apex of 152 it flattens somewhat at the apex distributing downward pressure to the sides. This relieves some pressure to the metatarsals directly above the apex of 152.

The placement of these embodiments in the forefoot just behind the metatarsals heads can provide a biomechanical solution similar to foam cushioning solutions currently in use for forefoot pain. These cushioning solutions, mentioned above, have built up areas of foam or rubbery material just behind the metatarsal heads for support of areas of pain at the metatarsal heads. The convex structures of embodiments in the forefoot of footwear could work with and/or replace the foam cushioned solutions.

It is understood 150, 151, 152, 153 can be applied to other footwear. It is understood portions 150, 151, 152, 153 can be portions of a shoe sole and function independently of portions 150A, 21, 51. Further, portions 150, 151, 152, 153 can be a part of a shoe sole without 150A, 21, 51.

Other footwear may include a convex form similar to 152 and strike plate platform similar to 153 without sides 153A, 153B. This may be placed in a different location along the length of the foot on the medial side. This is unlike the placement of FIGS. 32-33A which are placed across the width of the foot. This footwear may support the arch of the foot on the medial side that proceeds from the rear area of the foot forward. In footwear this structure may have components that join the rear foot and forefoot structures. Further, in this footwear and others with structures of the invention, these structures may be replaceable and adjustable to differing feet and amounts of downward pressure.

Other footwear-with a convex portion on top of a strike plate may also be placed in similar locations in the rear foot, forefoot and medial arch. This footwear may have an additional connection at the convex shape and/or strike plate to a third point farther towards the inner middle of the foot. This connector point is similar to a hinge that allows down and back up compression and tension while remaining relatively stationary. This third point connector hinge creates a somewhat horizontal triangulated shape to the structure of footwear. While the original vertical triangulation of the convex top and its stable bottom at the strike plate remains. In these and other footwear with a convex shape and strike plate the convex shape may be a dome or partial dome shape.

Further, two or more of these rear foot, medial arch and forefoot structures may be connected near or at their connected hinges. This can be for the purpose of the transfer of force to act upon the compression and directional force from the foot above. Further, as the greatest impact is at the rear foot, the rear foot embodiment alone may be a transmitter of force to another or other structures forward of it in the foot. Further, in some footwear curled springs in the rear foot convex sides may be added to or replace the sides.

It is understood the materials and configurations of footwear in the upper and sole may be of different qualities, but function within the utilization of the tension and compression of the invention. They may be physically connected or not while performing their functions.

It is understood the material of the embodiments of FIGS. 3-33A and alternates may be composed of material such that there may be a method of fabrication that would allow a consumer and/or professional to adjust the footwear to the consumer. Further, it is understood portions of embodiments that are in FIGS. 3-33A may be made of material such that the substantially horizontal width and substantially vertical angling of an embodiment that adjusts to the angle of the first and fifth metatarsal may be folded up vertically, or by another means by a foot professional or by a consumer in order to fit the footwear to both the width of the foot and the angle of the metatarsals. This custom fitting by a consumer or professional may eliminate the need in footwear for width sizing and angling adjustments by the manufacturer. Further, remaining excess portions or portions too large for the consumer's foot may be pre-scored to be snapped off or snipped off by a toenail clipper or other tool.

It will now be apparent to those skilled in the art that other embodiments, improvements, details, and uses can be made consistent with the letter and spirit of the foregoing disclosure and within the scope of this patent, which is limited only by the following claims, construed in accordance with the patent law, including the doctrine of equivalents.

What is claimed is:

1. A footwear article with one or more hard sole portions and one or more hard upper portions interacting with a sole portion, including an upper portion adjacent the medial first metatarsal to prevent or to limit foot shifting and/or spreading when a user's weight is applied to a sole area adjacent to and interactively connected to one of the at least one or more upper portions, the structure thereby sustaining the foot.

2. The article of claim 1 wherein a sole and multiple upper portions are provided including at the medial first metatarsal and at one or more other portions of the foot.

3. The article of claim 2 wherein multiple upper portions are arranged for managing triangulation in response to downward pressure of the foot on the sole in one or more of forefoot, arch and hind foot zones or across two or more of such zones.

4. The article of claim 2 wherein multiple upper portions are arranged in forefoot zones to manage triangulation without use of upper portions at the hind foot zone to manage such triangulation.

5. The footwear article of claim 1 as an integral part or parts of a shoe, boot, sneaker, sandal or like foot covering.

6. The footwear article of claim 1 as an insert for a shoe, boot, sneaker, sandal or like foot covering.

7. A footwear article comprising a sole and upper constructed and arranged such that downward pressure of a wearer's foot in forefoot motion causes reaction force vectors in the sole and upper to converge forward to transfer force on the medial side on or near a mid-foot joint and then a further following vector (20) to enable overall shifting of force in the footwear as the transfer of weight to the wearer's rear foot begins,
the footwear being constructed and arranged such that downward pressure of the foot and lateral to medial directional force on the foot in motion putting a vertical upper portion of the footwear in tension to maintain a foot portion in position and retain it.

8. The article of claim 7 wherein a sole and multiple upper portions are provided including at the medial first metatarsal and/or more other portions of the foot.

9. The article of claim 7 with multiple upper portions are arranged for managing triangulation in response to downward pressure of the foot on the sole in one or more of forefoot, arch and hind foot zones or across two or more of such zones.

10. The footwear article of claim 7 as an integral part or parts of a shoe, boot, sneaker, sandal or like foot covering.

11. The footwear article of claim 7 as an insert for a shoe, boot, sneaker, sandal or like foot covering.

12. The article of claim 12 comprising:
means forming a layer of bowed sole material to define a spanning convex region within a sole portion,
means forming a concave striker plate below the said spanning layer with side runners on both medial and lateral sides engaging edges of the spanning layer,
whereby downward pressure on the spanning layer by a user's foot buckles it downwardly into the convex region and imparts force to the side runners to spread the impact force and produce a reactive response force sustaining the foot.

13. The article of claim 12 where the spanning layer and strike plate are in a hind foot region and constructed and arranged to interact with a counter element.

14. The article of claim 13 comprising a counter element interactively engaged with the striker plate's runners.

15. The article of claim 12 whereby spanning layer and strike plate are in a forefoot region.

16. The article of claim 12 wherein combinations of spanning layer and strike plate are in both hindfoot and forefoot regions.

17. The method for managing triangulation of a human foot with a shoe, boot, sneaker, sandal or like article occurring through the process of a footstep by causing downward pressure of a wearer's foot in the article in forefoot motion (1) to initiate a process of triangulation in a way causing reaction force vectors in the sole and upper of the article to converge forward to transfer force from the lateral to a medial side of the article on or near one or more of the foot's mid-joints or following forefoot joints and then developing a further following reaction force vector to enable pivoting overall shifting of force in the article as the transfer of weight to the wearer's rear foot begins,
doing so in a way using sole and upper vertical constraints to utilize downward pressure of the foot and lateral to medial directional force on the foot in motion that puts one or more upper vertical constraints in tension to maintain a foot portion in position and sustain it.
